(19) United States
(12) Patent Application Publication
(10) Pub. No.: US 2023/0270925 A1
(43) Pub. Date: Aug. 31, 2023

(54) BLOOD DIALYZING APPARATUS AND METHOD

(71) Applicant: Exorenal Inc, Baltimore, MD (US)

(72) Inventor: Jake Kyungsoo Lee, Ellicott City, MD (US)

(21) Appl. No.: 17/893,149

(22) Filed: Aug. 22, 2022

Related U.S. Application Data
(63) Continuation-in-part of application No. 16/703,757, filed on Dec. 4, 2019.
Application No. 16/703,757 is a continuation-in-part of application No. 17/082,016, filed on Oct. 28, 2020.
(60) Provisional application No. 63/235,163, filed on Aug. 20, 2021.
(60) Provisional application No. 63/256,023, filed on Oct. 15, 2021.

(51) Int. Cl. A61M 1/16; A61M 1/26

(52) U.S. Cl. CPC A61M 1/1621; A61M 1/267; A61M 2205/3334

(57) ABSTRACT

Provided is a blood dialyzing apparatus having multiple fluid chambers each having an internal space, a chamber pressurizing member compressing or expanding the internal spaces of the chambers, a chamber pressurizing member driver driving the chamber pressurizing member, and a flow control unit. The chambers are each connected with a first flow tube through which a fluid is provided to the chamber and a second flow tube through which a fluid of the chamber is discharged therefrom. The flow control unit controls a flow through the flow tubes connected to the multiple fluid chambers.

### TECHNICAL FIELD

The present invention relates to blood dialyzing apparatus and method, in which a plurality of blood chambers are compressed and expanded simultaneously to allow blood or dialysis fluid to flow through a blood dialyzing filter, thereby making the apparatus simplified and light-weighted, providing easy operation, reducing cost for the dialyzing treatment, and eventually enabling the dialyzing treatment to be conducted at home.

### BACKGROUND

When there is a kidney dysfunction, water and waste products that have to be discharged out of body accumulate in blood and imbalance of electrolytes in the body occurs. Most commonly performed to improve such a kidney failure symptom, is hemodialysis which is to circulate blood out of body and rid the blood of the accumulated uremic toxin and excess water by a semi-permeable dialysis membrane. Hemodialysis is a method of seeking an electrolyte balance and ridding the body fluid of uremic toxin and excess water, taking advantages of diffusion applied due to the concentration difference and filtration applied due to the pressure difference between blood and dialysis fluid.

Hemodialysis is the example of the blood dialyzing treatment in which blood of a patient is circulated extracorporeally to remove toxic substances from or supply beneficial ingredients to the blood. The blood dialyzing treatment is frequently combined with a blood dialyzing filter in which mass transfer between blood (i.e., a physiologic body fluid) and dialysis fluid (i.e., a purified sterile solution).

Most commonly used of blood dialyzing filter is the type that is a cylinder-shaped container charged with a bundle of hollow fiber membranes and port-processed at both ends thereof by use of a synthetic resin like polyurethane. It is because the hollow fiber blood dialyzing filter has excellent mass-transfer efficiency resulting from large effective surface area between blood and dialysis fluid compared to the small size as a whole.

Conventional blood dialyzing devices require a balancing unit connected to the multiple dialysis fluid tubes, two or more dialysis fluid pumps to transfer dialysis fluid, and a blood pump to transfer blood of a patient. It is also indispensable to disinfect the balancing unit, the dialysis fluid pumps, and the dialysis fluid flowing tubes on a regular basis, rendering the conventional blood dialyzing unit complex in the structure and complicated to use.

U.S. Pat. No. 4,054,522 discloses a degassing apparatus employing multiple fluid chambers, through which dialysate sequentially flows and therefore, functioning as a dialysate pump. The fluid chambers are pressurized or expanded by the piston pushing a rolling diaphragm. In addition, the diaphragm pump having four diaphragm chambers is used to transfer blood, according to WO 8,601,115, in which the diaphragm chambers are operated by a pneumatic driver. However, despite using multiple fluid chambers, they are neither able to achieve the balancing accuracy nor regulate the net volume removal. Additional separate ultrafiltration pumps or balancing chambers are required.

The prior disclosures only focus on transferring the fluid, such as either blood or dialysis fluid, and therefore, they are limited to using the diaphragm type pumps. Unlike the disclosure, the four diaphragm chambers are not able to ensure blood flow rates equal upstream and downstream of the dialyzer due to flow resistances through the arterial blood circuit.

In order to solve the aforementioned problems, a novel blood dialyzing apparatus is provided, in which multiple blood chambers are compressed and expanded to transfer blood. The multiple chambers ensure blood flow rates upstream and downstream of the blood dialyzing filter to be regulated independently. Neither separate blood pump nor a separate ultrafiltration pump, nor a separate balancing chamber are required. Therefore, the entire system can be sufficiently miniaturized and light-weighted, and easy to be installed while reducing the cost for blood dialyzing treatment. The blood dialyzing apparatus will eventually be an optimal alternative for the blood dialyzing treatment in a place out of hospitals.

### SUMMARY

The blood dialyzing apparatus according to an embodiment of the present invention is configured to include a plurality of chambers each having an internal space, chamber pressurizing members compressing or expanding the internal spaces of the chambers, a chamber pressurizing member driver operating the chamber pressurizing members, and a flow control unit. In particular, the blood dialyzing apparatus may be configured to include first to fourth chambers each connected with an inflow tube and an outflow tube, where a fluid is provided to the chamber through the inflow tube and the fluid of the chamber is discharged therefrom through the outflow tube.

The fluid is supplied to the blood dialyzing filter through the first and second chambers and the fluid of the blood dialyzing filter is discharged therefrom through the third and fourth chambers. That is, the first and second chambers are the supplying chambers while the third and fourth chambers serve as the discharging chambers. Accordingly, the outflow tubes of the first and second chambers are connected to the blood dialyzing filter and the inflow tubes of the third and fourth chambers are connected to the blood dialyzing filter.

Here, the flow control unit opens or blocks a flow through the inflow and outflow tubes connected to the first to fourth chambers.

One of the supplying chambers is compressed when the other supplying chamber is expanded. One discharging chamber is compressed and the other discharging chamber is expanded. Here, an expanded volume of the supplying chamber may remain equal to or greater than a compressed volume of the supplying chamber. Similarly, an expanded volume of the discharging chamber may remain equal to or greater than a compressed volume of the discharging chamber.

The operating of the blood dialyzing apparatus may involve (S10) unblocking the outflow tube of the first chamber, the inflow tube of the second chamber, the outflow tube of the third chamber and the inflow tube of the fourth chamber, (S21) compressing the first chamber to discharge the fluid therein to the blood dialyzing filter, (S22) expanding the second chamber to allow the fluid to flow therein, (S23) compressing the third chamber to discharge the fluid therefrom, and (S24) expanding the fourth chamber to allow the fluid of the blood dialyzing filter to flow into the chamber. In addition, S21 to S24 may occur substantially at the same time, taking 1.5 to 6.5 seconds.

Disclosed is the blood dialyzing apparatus, in which multiple blood chambers are compressed and expanded to transfer blood or dialysis fluid. The multiple chambers ensure blood or dialysis fluid flow rates upstream and downstream of the blood dialyzing filter to be regulated independently. Neither separate blood pump nor a separate ultrafiltration pump, nor a separate balancing chamber are required. Therefore, the entire system can be sufficiently miniaturized and light-weighted, and easy to be installed while reducing the cost for blood dialyzing treatment.

### BRIEF DESCRIPTION OF THE DRAWINGS

The accompanying drawings are included to provide a further understanding of the present invention and are incorporated in and constitute a part of this specification. The drawings illustrate exemplary embodiments of the present invention and, together with the description, serve to explain principles of the present invention. In the drawings:

FIGS. 1A and 1B are views illustrating a schematic diagram of a blood dialyzing apparatus according to an embodiment of the present invention;

FIGS. 2A to 2C are views illustrating a circuit diagram of a blood dialyzing apparatus according to an embodiment of the present invention;

FIGS. 3A and 3B are views illustrating a fluid pumping unit of a blood dialyzing apparatus, including cylinder-shape chambers and piston-shaped chamber pressurizing members according to an embodiment of the present invention;

FIG. 4 is a view illustrating a fluid pumping unit of a blood dialyzing apparatus, including fluid sacs and pneumatic sac pressurizing channels according to an embodiment of the present invention;

FIG. 5 is a view illustrating a blood dialyzing filter according to an embodiment of the present invention;

FIG. 6 is a view illustrating a flow control unit formed of a pressurizing type valve;

FIGS. 7 and 8 are views illustrating a flow control unit formed of a rotating type valve;

FIGS. 9 and 10 are views illustrating an operation of a blood dialyzing apparatus according to an embodiment of the present invention, in which the chambers serve as a means of transferring blood through the blood dialyzing filter;

FIG. 11 is a flowchart illustrating a method of operating a blood dialyzing apparatus according to an embodiment of the present invention;

FIGS. 12 to 20 are views illustrating steps of operating a blood dialyzing apparatus as described in FIG. 11 according to an embodiment of the present invention;

FIGS. 21 to 24 are views illustrating modified steps of operating a blood dialyzing apparatus according to an embodiment of the present invention.

### DETAILED DESCRIPTION OF PREFERRED EMBODIMENTS

Preferred embodiments of the present invention will be described below in more detail with reference to the accompanying drawings. The present invention may, however, be embodied in different forms and should not be constructed as limited to the embodiments set forth herein. Elements and components disclosed in the drawings may be exaggerated or simplified to improve the clarity and convenience of the description. Terms or languages defined in the present disclosure may have different meaning according to the users’ intention or practice. These terms should be interpreted as a meaning corresponding to the technical concept of the present invention disclosed throughout the specification of the present invention.

As used herein, the singular forms “a,” “an,” and “the” are intended to include the plural forms as well, unless the context clearly indicates otherwise. In addition, the expressions defining the relationship of elements or components should be interpreted as broad as possible. For example, it will be understood that when an element or layer is referred to as being “on,” “connected to,” “coupled to,” or “adjacent to” another element or layer, it can be directly on, connected, coupled, or adjacent to the other element or layer, or intervening elements or layers may be present therebetween. It will also be understood that when an element is same or identical to another element, the element can be completely same or identical to another element, or it includes that the two elements may be “substantially” similar to each other. In the same manner, for the expression showing the equivalence of time such as “simultaneously” or “at the same time,” it should be understood that it happens completely at the same time, or they may happen at substantially the similar time. The same reference denotations may be used to refer to the same or substantially the same elements throughout the specification and the drawings.

Hereinafter, the blood dialyzing apparatus will be described in detail with reference to the accompanying drawings.

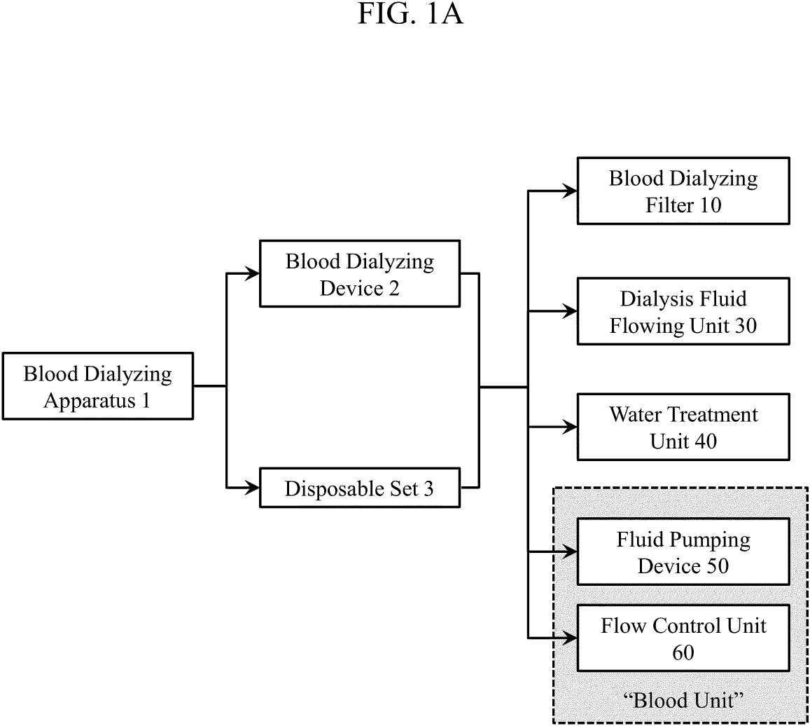

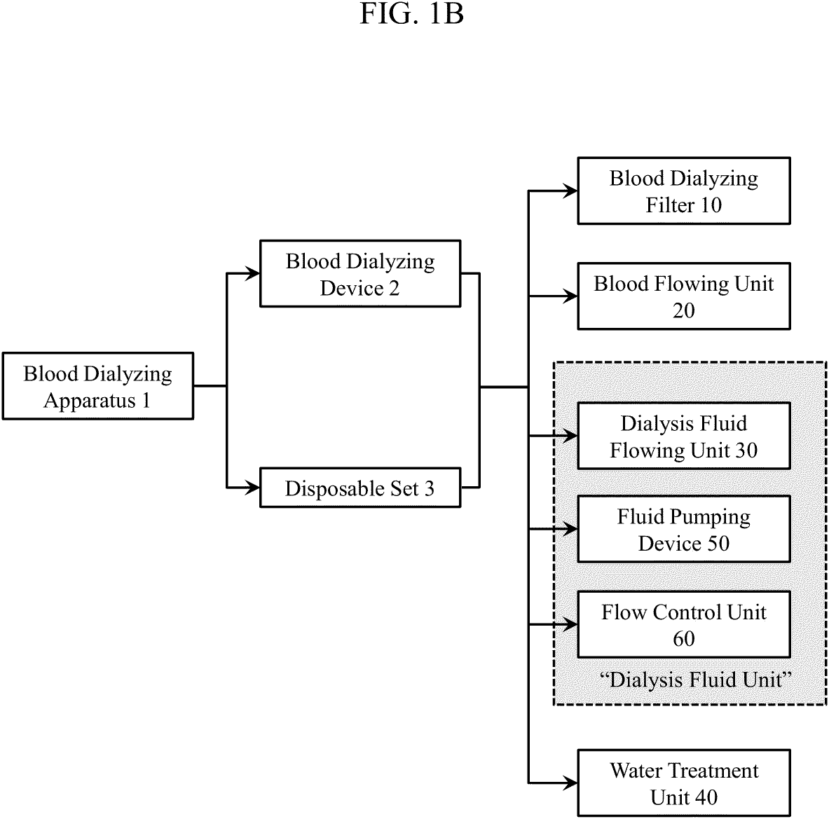

FIG. 1 is a schematic diagram of a blood dialyzing apparatus. Exemplary blood dialyzing apparatus may include the device to preserve blood, separate blood cells or plasma from whole blood, dialyze blood of a patient with renal failure (acute or chronic), or detoxify blood for patients with liver failure (acute or acute-on-chronic) or multi-organ failure. That is, any devices to replace impaired functions of lung, heart, liver or kidney may be used as the blood dialyzing apparatus according to an embodiment of the present invention.

The blood dialyzing apparatus 1 is configured to include a blood dialyzing device 2 and a disposable set 3. The blood dialyzing device is a hardware unit with a case in which various electric elements are mounted to perform the treatment. Software and programming to run the electric elements are installed. The disposable set is a consumable element used for each treatment. For example, the disposable unit includes tubes through which blood, dialysis fluid, or any biologic fluid flows, air drip chambers to remove air bubbles, and/or a blood dialyzing filter.

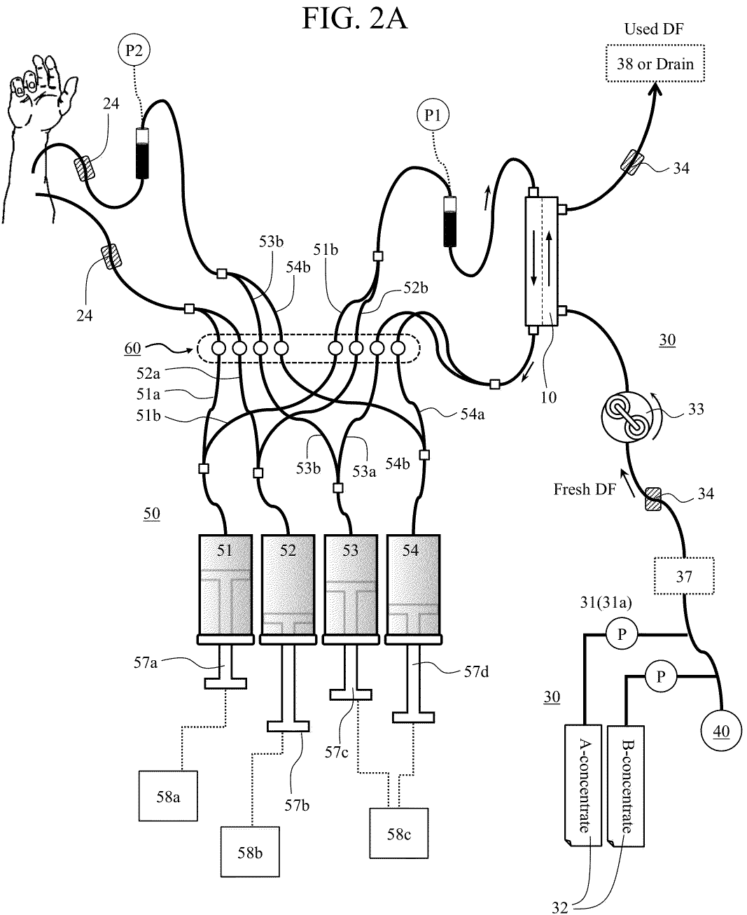

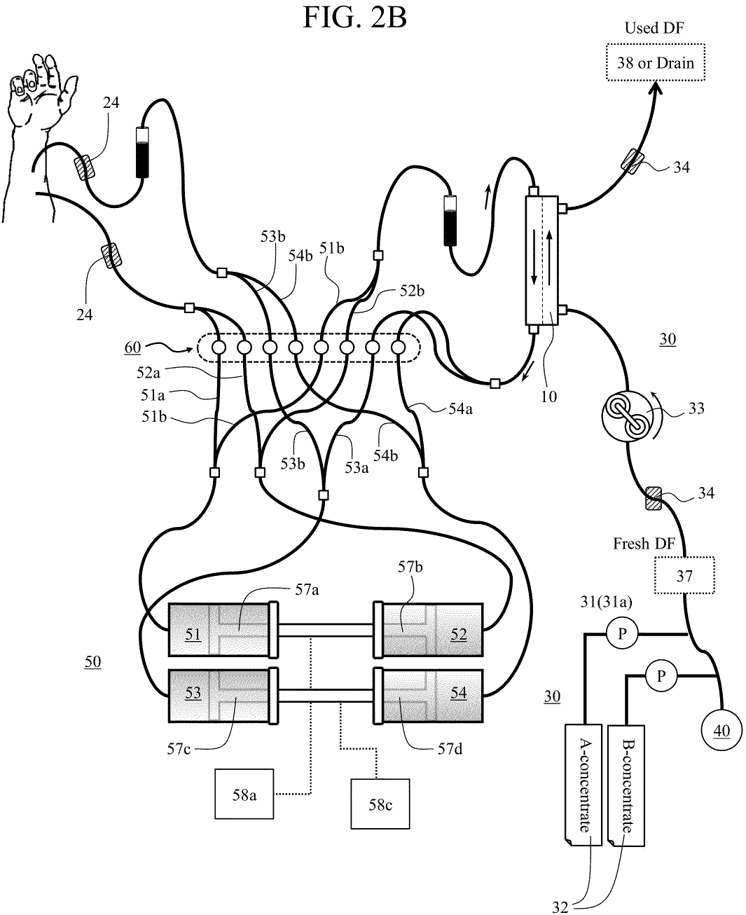

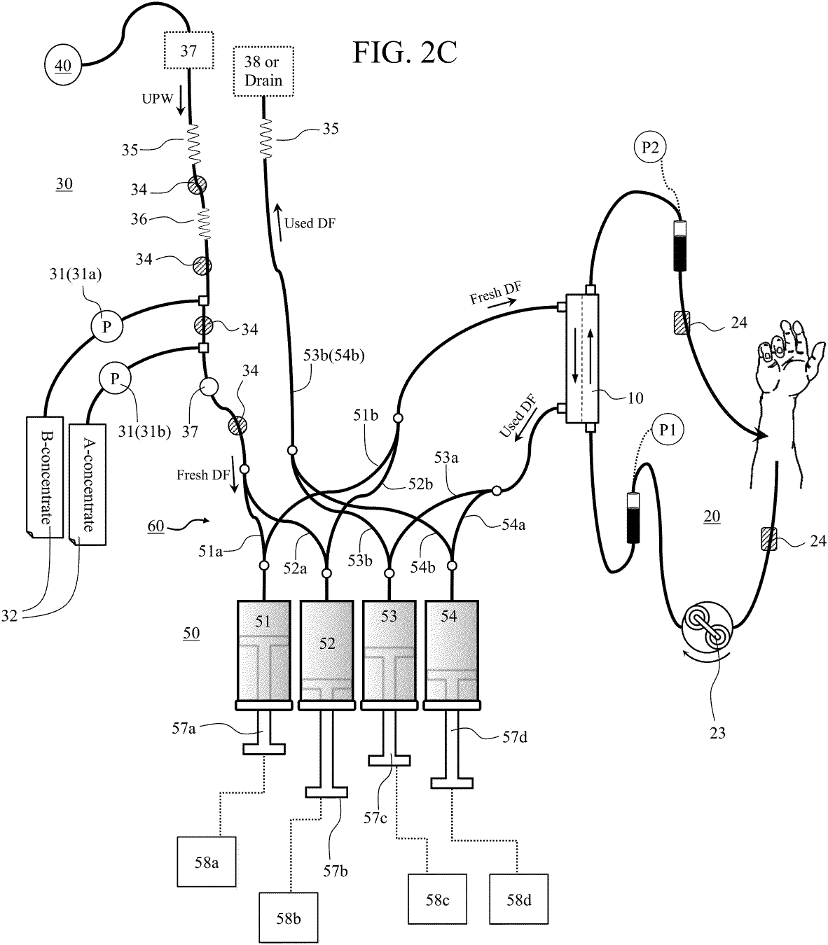

FIGS. 2A and 2B illustrate circuit diagrams of the blood dialyzing apparatus 1 according to an embodiment of the present invention. The blood dialyzing apparatus 1 includes a dialysis fluid flowing unit 30 where fresh dialysis fluid is prepared by adjusting ion balance and then transferred through a blood dialyzing filter 10, a water treatment unit 40 generating ultrapure water, a fluid pumping unit 50 transferring blood, and a flow control unit 60 controlling flow passages through the blood flowing tubes. Various safety and monitoring sensors 24 and 34 may also be provided to monitor the blood dialyzing treatment. The blood dialyzing apparatus 1 further includes the blood dialyzing filter 10 in which blood is dialyzed. Mass transfer occurs between blood and dialysis fluid in the blood dialyzing filter 10.

The blood dialyzing apparatus 1 according to an embodiment of the present invention is not limited to the structure shown in FIGS. 2A and 2B. As shown in FIG. 2C, the blood dialyzing apparatus 1 may include a blood flowing unit 20 where blood is transferred through the blood dialyzing filter 10, a water treatment unit 40 generating ultrapure water, a fluid pumping unit 50 transferring dialysis fluid, and a flow control unit 60 controlling flow passages through the dialysis fluid flowing tubes.

Referring to FIGS. 2A to 2C, the fluid pumping unit 50 includes a plurality of fluid chambers each having an internal space, a chamber pressurizing member 57 compressing or expanding the internal spaces of the fluid chambers, and a chamber pressurizing member driver 58 operating the chamber pressurizing member 57.

Specifically, the fluid pumping unit 50 according to an embodiment of the present invention may be configured to include four fluid chambers, i.e., first to fourth fluid chambers 51 to 54. The chamber pressurizing member 57 may be configured to further include first to fourth chamber pressurizing members 57a to 57d, which are respectively disposed inside each of the fluid chambers 51 to 54 to compress or expand the respective chamber.

Here, although the term ‘dialysis fluid’ is used to distinguish it from blood, the dialysis fluid is not limited to the fluid that is used for hemodialysis, continuous renal replacement therapy (CRRT), or peritoneal dialysis. The dialysis fluid may be any fluids that can be used for any types of treatments requiring extracorporeal blood circulation, including but not limited to plasma, serum, distilled water, isotonic saline solution, lactose solution, and the like.

Each of the fluid chambers 51 to 54 is connected with inflow and outflow tubes. Fluid such as blood or dialysis fluid is supplied to the chamber through the inflow tube and the fluid is removed from the chamber through the outflow tube. For example, the first chamber 51 is connected with the first chamber inflow tube 51a and the first chamber outflow tube 51b. Blood or dialysis fluid flows into the first chamber 51 through the first chamber inflow tube 51a and blood or dialysis fluid inside the first chamber 51 leaves through the first chamber outflow tube 51b. Similarly, the second chamber 52 is connected with the second chamber inflow tube 52a and the second chamber outflow tube 52b.

The inflow and outflow tubes are merely expressions to describe the tubes connected to the chamber and they shouldn’t be interpreted that a fluid must flow into the chamber through the inflow tube or leave the chamber through the outflow tube. For example, a fluid flows into the chamber through the outflow tube, or a fluid may be provided to (or discharged from) the chamber through both inflow and outflow tubes. In addition, as shown in FIGS. 2A to 2C, each chamber is connected with the inflow and outflow tubes, but they may overlap in a portion such that a single tube is connected to the chamber.

The first to fourth chambers 51 to 54 may be compressed or expanded simultaneously. All of the four chambers may be compressed, or expanded simultaneously. Alternatively, some of the chambers are compressed while the other chambers are expanded. For example, two chambers are expanded while the other two chambers are expanded, which occurs simultaneously. Three chambers are compressed while one chamber is expanded, and vice versa.

The chamber pressurizing members 57a to 57d are operated by the chamber pressurizing member driver 58. According to an embodiment of the present invention, each of the chamber pressurizing members 57a to 57d may be operated by a separate chamber pressurizing member drivers 58a to 58d, resulting in the independent operation of each chamber pressurizing member. For example, the first chamber pressurizing member 57a is operated by a first chamber pressurizing member driver 58a and the second chamber pressurizing member 57b may be run by a second chamber pressurizing member driver 58b, and so on.

Alternatively, two or more chamber pressurizing members may be run by a single chamber pressurizing member driver. In FIG. 2B, the first and second chamber pressurizing members 57a and 57b are illustrated to operate by a single chamber pressurizing member driver 58a, and the third and fourth chamber pressurizing members 57c and 57d also operate by a chamber pressurizing member driver 58c. Thus, the first and second chamber pressurizing members 57a and 57b may be formed as a single body, and the third and fourth chamber pressurizing members 57c and 57d may be formed in one body.

In addition, all the chamber pressurizing members 57a to 57d may be operated by a single chamber pressurizing member driver 58 according to an embodiment of the present invention.

The chamber pressurizing member driver 58 includes various structures which allow the chamber pressurizing members 57a to 57d to reciprocate along a straight line (or a curved line) so as to compress or expand the internal spaces of the chambers. An exemplary chamber pressurizing member driver may include a cam pushing the chamber pressurizing member 57 in a rectilinear direction and a motor rotating the cam. Alternatively, the chamber pressurizing member driver 58 may have a structure including a motor, a circular gear rotating by the motor, a linear gear moving along a straight line due to the rotation of the circular gear. Due to the rotation of the cam or circular gear, the chamber pressurizing member 57 moves along a rectilinear direction, and when the motor rotates further or rotates in an opposite direction, the chamber pressurizing member 57 may move to an opposite direction.

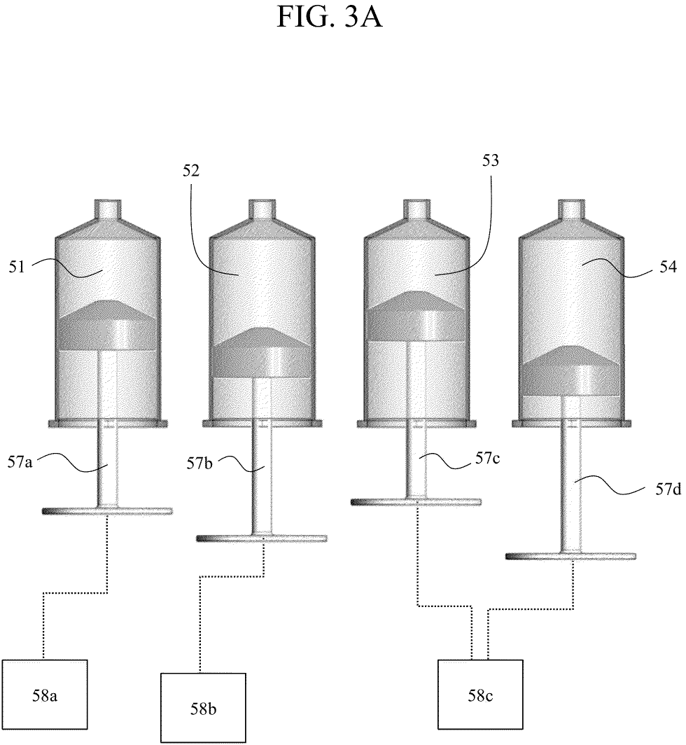

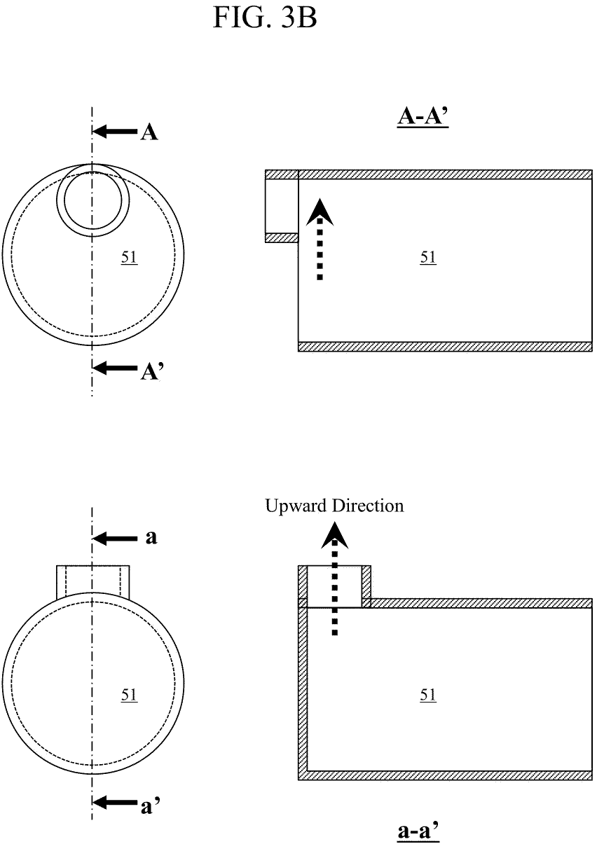

Referring to FIGS. 3A and 3B, the chambers according to an embodiment of the present invention may be configured to have a cylinder-shaped internal space and the chamber pressurizing members 57a to 57d have a piston shape, reciprocally and detachably disposed inside the cylinder-shaped chambers. The chambers may be made of a substantially inflexible material having a predetermined shape, such as plastic, polycarbonate, polyurethane, metallic material, etc. The chamber pressurizing members 57 preferably have a portion that is made of a substantially flexible material such as rubber, polymer, silicone, and the like.

However, the chamber and the chamber pressurizing member are not limited to the aforementioned structure. A container having an internal space to accommodate a fluid and any means that pressurizes or expands the internal space of the container to thereby make a fluid to flow through the container can be used as the chamber and the chamber pressurizing member. Exemplary chamber may include a fluid sac, a fluid bag, or a fluid tube that are flexible, and any means pressurizing or expanding the flexible fluid sac, fluid bag or fluid tubes can be used as the chamber pressurizing member. In this case, the chamber pressurizing member may preferably have a portion that is inflexible to compress the flexible chambers.

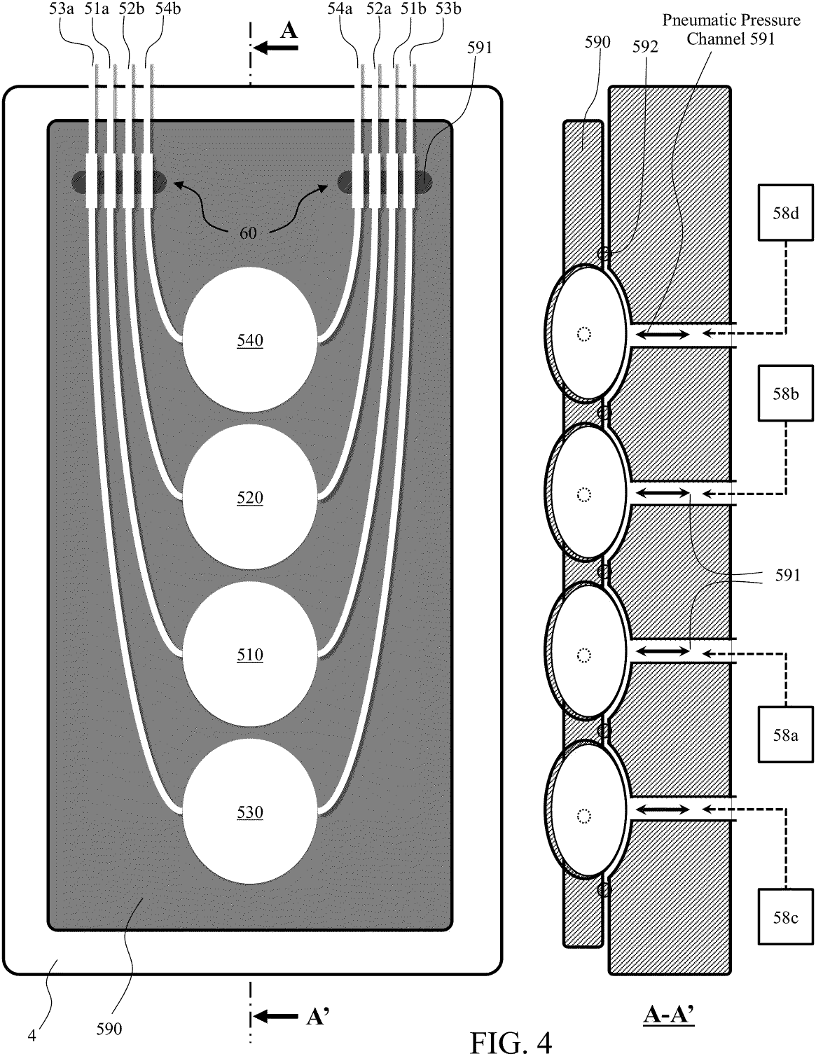

In an embodiment, FIG. 4 illustrates the fluid pumping unit 50, in which the fluid chambers have a form of a fluid sac 510 to 540 made of a flexible material that easily contracts and expands. The sacs are preferably installed inside a frame 590 as the frame 590 provides an installation space. The chamber pressurizing member 57 pressurizes or depressurizes the fluid sacs 510 to 540. For example, the fluid sacs may be compressed or expanded by an operation of a pneumatic driver, such as a pneumatic pump, gas pump, vacuum pump, and others. The pneumatic driver placed in the case compresses or decompresses the pneumatic channel 591, resulting in the compression or decompression of the fluid sacs. The pneumatic channel 591 may be able to serve as the chamber pressurizing members. A gasket 592 may be provided to prevent a leakage around the fluid sacs, such as plastic, polymer, silicone, metal, and others.

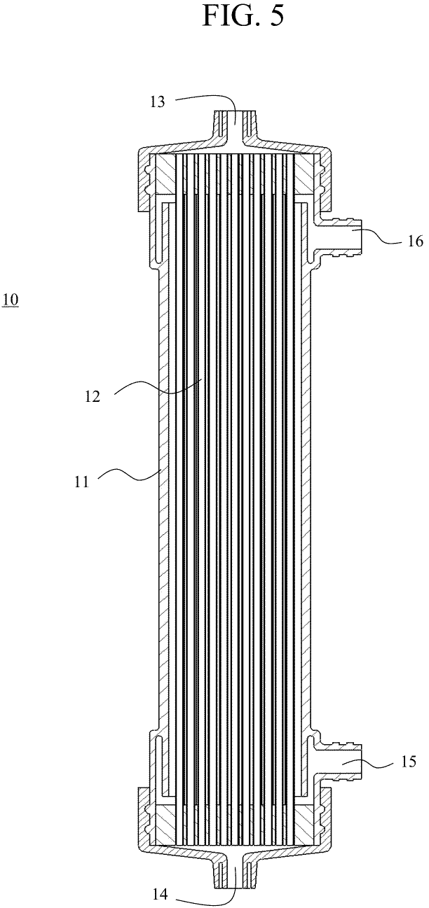

The blood dialyzing filter 10 includes various filter apparatuses to dialyze blood of a patient. As shown in FIG. 5, the blood dialyzing filter 10 may have a form in which a blood dialyzing membrane 12 is accommodated in the filter housing 11. The internal space of the filter housing 11 can be divided into multiple flow regions by the membrane 12, through which a separate fluid flows. In an embodiment, the blood dialyzing filter 10 is divided into a blood flow region and a dialysis fluid flow region by the blood dialyzing membrane 12.

The filter housing 11 is provided with a first blood port 13 and a second blood port 14 disposed at an opposite side thereof. Blood may enter the blood dialyzing filter 10 through the first blood port 13 and leave therefrom through the second blood port 14. Blood tubes 21 and 22 may be connected to the blood ports 13 and 14, respectively, to allow blood to flow through blood dialyzing filter 10. Also, a first dialysis fluid port 15 and a second dialysis fluid port 16 may be provided on the filter housing 11 to allow the dialysis fluid to flow through the blood dialyzing filter 10. Specifically, dialysis fluid may be provided to the blood dialyzing filter 10 through the first dialysis fluid port 15 and is discharged therefrom through the second dialysis fluid port 16.

Blood passes through the blood flow region inside the blood dialyzing filter 10 and dialysis fluid passes through the dialysis fluid flow region. Blood and dialysis fluid may be desirably configured to flow in the opposite directions to each other. The blood dialyzing filter 10 is not limited to the structure shown in the drawing, and may be modified into other forms including a hemodialyzer, an adsorption filter column, or a hemodiafilter.

Fresh dialysis fluid is produced in the dialysis fluid flowing unit 30, which is then transferred through the dialysis fluid circuit, as illustrated in FIGS. 2A to 2C. Acid and bicarbonate ion solutions (or acid and bicarbonate powder) are mixed with ultrapure water. Through this process, ion concentrations such as bicarbonate, sodium, etc., and pH of the dialysis fluid can be adjusted. The dialysis fluid flowing unit 30 may include dialysis fluid processing pumps 31 to transfer the acid and/or bicarbonate solutions 32. The dialysis fluid processing pumps 31 may further include first and second dialysis fluid processing pumps 31a and 31b to transfer the acid and bicarbonate solutions 32. Since the dialysis fluid processing pump 31 needs to deliver the precise amount of solutions, a precise metering pump may be used for the dialysis fluid processing pump 31. Exemplary dialysis fluid processing pump 31 includes a rotary piston pump, a metering peristaltic pump, a precise piston pump, and the like.

A fresh dialysis fluid container 37 and a used dialysis fluid container 38 may be used to store fresh dialysis fluid or to collect used dialysis fluid, respectively. However, fresh dialysis fluid can be supplied to the blood dialyzing filter 10 without being stored in the fresh dialysis fluid container 37 and the used dialysis fluid may be discarded without being collected in the used dialysis fluid container 38.

The dialysis fluid is not limited to be produced through the dialysis fluid flowing unit 30. The dialysis fluid may be provided by using a pre-made dialysis fluid bag. In addition, the blood dialyzing apparatus 1 may further be provided with dialysis fluid sensors 34 to measure the purity of the fresh dialysis fluid, such as a conductivity sensor.

In addition, a dialysis fluid pump 33 may be provided to transfer dialysis fluid. Various displacement pumps can be used as the dialysis fluid pump, including a peristaltic pump, a roller pump, a piston pump, a rotary piston pump, and so on.

The water treatment unit 40 generates ultrapure water and includes multiple filtration stages, such as a pre-processing filter, a carbon filter, a reverse osmosis filter, ionexchange resin beds, and/or an endotoxin retention filter. The water treatment unit 40 can be modified into a different configuration to prepare ultrapure water satisfying the requirement of the blood dialyzing treatment.

The flow control unit 60 controls flow through the inflow and outflow tubes. Various valve structures that can open or close the flowing tubes may be used, such as a one-way valve, a solenoid valve, an on-off valve, a pressurizing type valve, a rotating type valve, a pneumatic valve, or a combination of these valve types.

One-way valves ensure a fluid to flow in one direction. Solenoid valves and on-off valves may be installed on each of the flow tubes to open or block a flow therethrough. The pneumatic valve or a pneumatic valve assembly (including a pneumatic driver and a pneumatic channel) pressurizes or depressurizes a pneumatic channel, thereby compressing or decompressing, i.e., blocking or opening, the flow tubes through which the flow control unit 60 controls a flow. Exemplary pneumatic flow control unit 60 is illustrated in FIG. 4. Various types of pneumatic drivers can be used to pressurize or depressurize the pneumatic channel.

The flow control unit 60 opens or blocks eight flow tubes 51a, 51b, 52a, 52b, 53a, 53b, 54a and 54b. Specifically, the flow control unit 60 according to an embodiment of the present invention blocks the tubes 51a, 52b, 53a, 54b and the tubes 51a, 52b, 53a, 54b in an alternate manner.

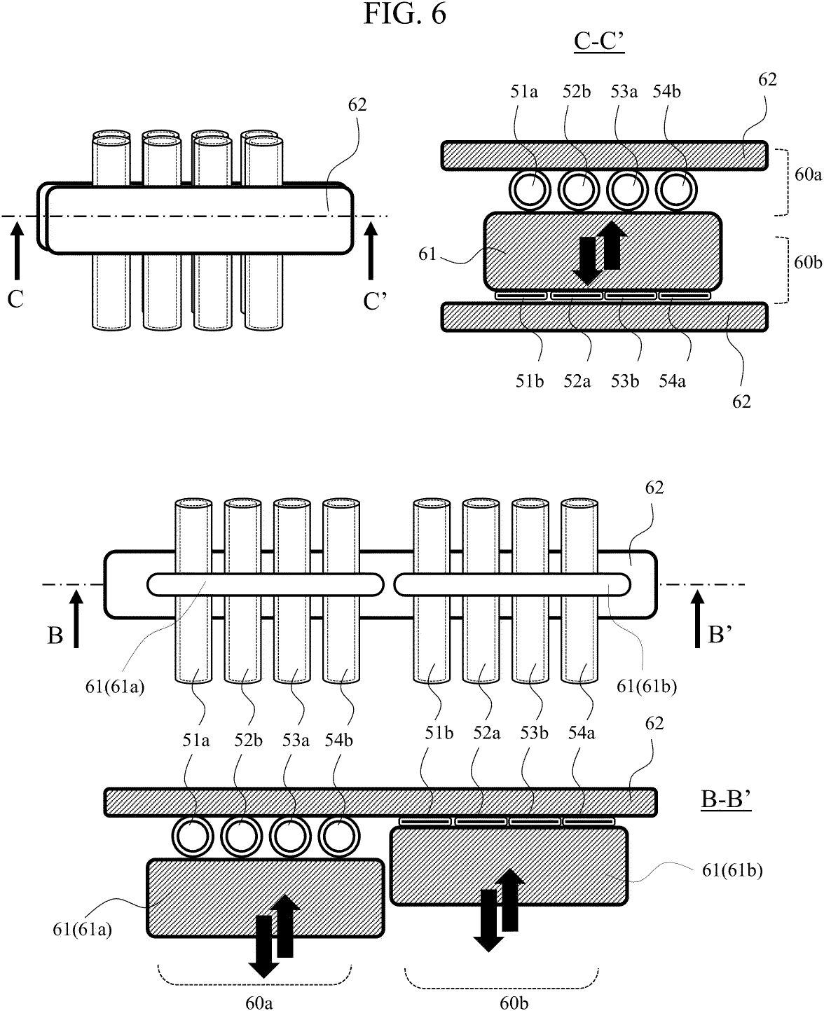

FIG. 6 illustrates the pressurizing type valve for the flow control unit 60. The pressurizing type valve includes a flow blocking member 61 reciprocating in a straight line (or in a curved line) to compress a portion of the tubes through which the flow control unit 60 controls a flow, a flow blocking wall 62 supporting the tubes compressed by the flow blocking member 61, and a flow blocking member driver providing a straight or curved force to the flow blocking member 61.

When the flow blocking member 61 moves to the tubes 51a, 52b, 53a and 54b, an end of the flow blocking member 61 compresses the tubes 51a, 52b, 53a and 54b supported by the flow blocking wall 62 and blocks the flow therethrough. At this time, the flow passages through the tubes 51b, 52a, 53b and 54a are opened. Similarly, the flow blocking member 61 moves to the tubes 51b, 52a, 53b and 54a, and another end of the flow blocking member 61 compresses the tubes supported by the flow blocking wall 62 and blocks the flow therethrough. Therefore, the flow control unit 60 is configured to block a half, or at least a half, of the flow tubes through which the flow control unit 60 controls the flow passages.

The flow control unit 60 may control the flow passages through the tubes 51a, 51b, 52a, 52b, 53a, 53b, 54a, and 54b using two or more flow blocking members 61a and 61b, as shown in FIG. 6. In this case, two or more flow blocking member drivers may be used to operate each of the flow blocking members 61a and 61b.

Alternatively, when the tubes are made of flexible materials, such as rubber, silicone, polyurethane, polyacetate, other polymers, etc., it may be possible to bend the flow tubes by a predetermined angle to thereby block the flow passage through the flow tubes. The flow blocking member 61 may not only compress the tubes to close the flow inside, but also bend the tubes to block the flow.

The flow blocking member driver includes various structures that can apply a reciprocating movement force (that is, for a rectilinear or curvilinear movement) to the flow blocking member 61. Substantially the same description for the chamber pressurizing member driver 58 can be applied to the flow blocking member driver.

For example, the flow blocking member driver may include a cam for pushing the flow blocking member 61 toward the flow blocking wall 62 supporting the tubes and a motor rotating the cam. When the flow blocking member 61 compresses the tubes due to the rotation of the cam, the flow therethrough may be blocked. When an external force by the cam is removed, the flow blocking member 61 may detach from the tube, and the tube may be restored to the original state, expanding the inside of the tube. Alternatively, an eccentric cam connected to a motor may rotate and compress one side of the tube and block the flow therethrough. The cam further rotates such that an external force applied by the cam may be removed and the tube is restored to its original status, expanding the inside of the tube.

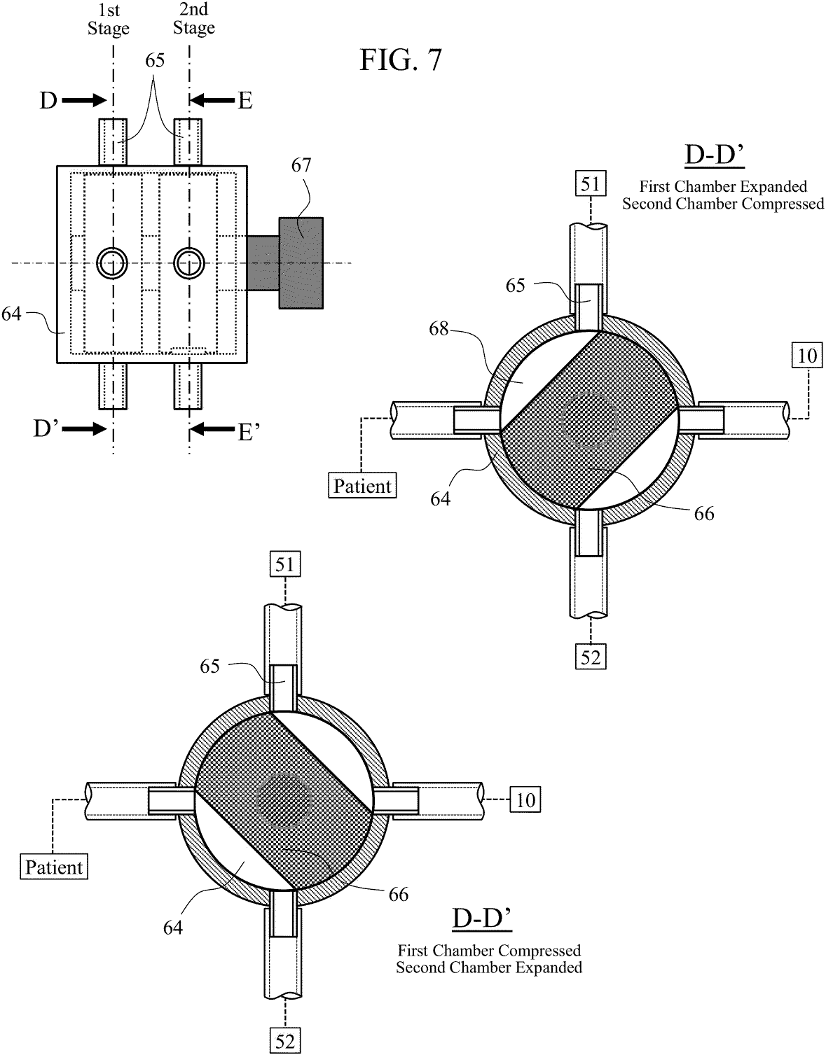

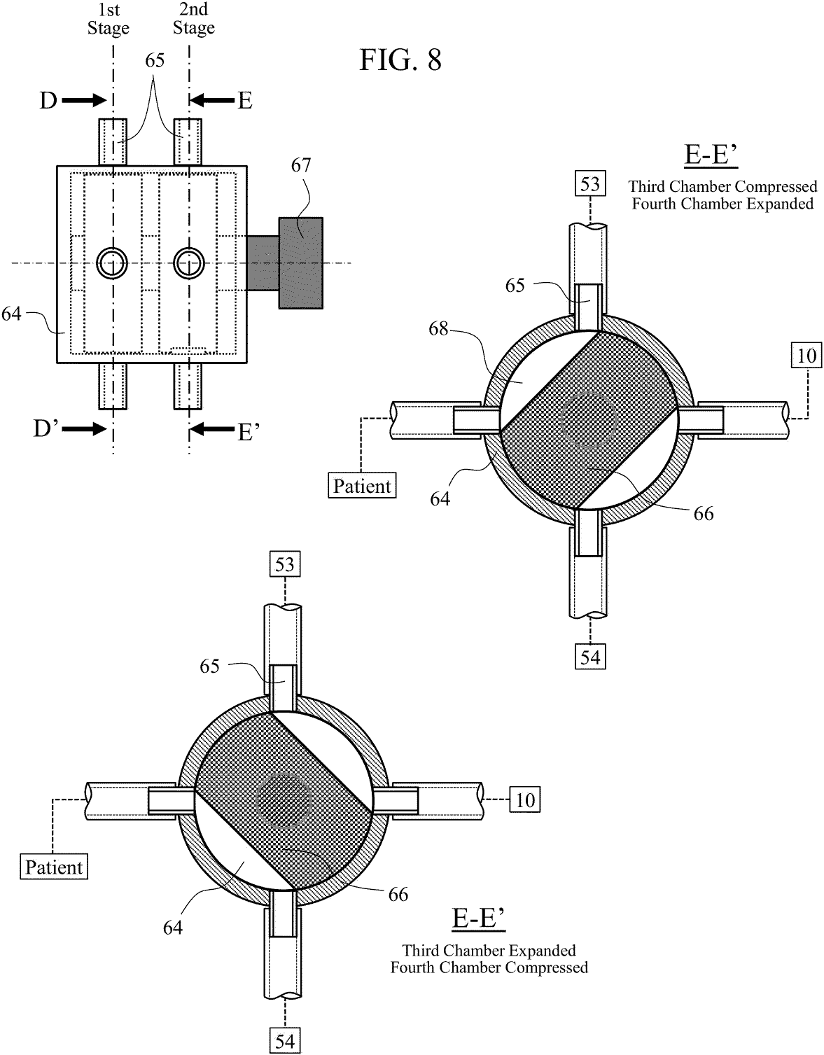

As illustrated in FIGS. 7 and 8, the flow control unit 60 can be modified into a structure having the rotating type valve. The rotating type valve includes a flow control housing 64 having an internal space, a flow control rotor 66 which is disposed inside the flow control housing 64, a plurality of flow control ports 65 disposed on the flow control housing 64 and penetrating the flow control housing 64, and a rotor driver 67 operating the flow control rotor 66.

The flow control rotor 66 and the internal space of the flow control housing 64 are preferably cylinder-shaped, allowing the flow control rotor 66 to easily rotate inside the flow control housing 64. However, the flow control rotor 66 may be modified so as to move along a rectilinear direction. The flow control rotor 66 may also be able to rotate while moving along a rectilinear direction. Due to the rotation (and/or linear movement) of the flow control rotor 66, a flow passage can be connected between at least two flow control ports 65.

The flow control rotor 66 may be formed with a recessed portion 68 to make it easier for a fluid to flow through two adjacent flow control ports 65. The recessed portion 68 may have a cross-sectional shape of a crescent moon, a rectangular, a square, a quadrilateral, or a triangular shape.

The flow control ports 65 formed in the flow control housing 64 may be spaced apart along a circumferential direction of the internal space of the flow control housing 64. In addition, the flow control ports 65 may be configured to face the cylinder surface of the rotor 66 as shown in the drawings.

The flow control rotor 66 rotates unidirectionally or bidirectionally to control the opening and blocking of the flow passage through the flow control ports 65. However, as aforementioned, the flow control rotor 66 can move along a rectilinear direction or rotate while moving along a rectilinear direction. The time for opening or blocking the flow passage can be controlled by regulating the movement speed of the flow control rotor 66.

In a preferred embodiment, the flow control rotor 66 needs to be tightly attached to the inner surface of the flow control housing 64 to inhibit a leakage through the contact surface of the flow control rotor 66 and the flow control housing 64. Thus, the flow control rotor 66 and the flow control housing 64 can be made of a material that can prevent a fluid from passing through the contact surface such as polymer, plastic, metallic substance, ABS, acrylic, or the like. Alternatively, in order to prevent a leakage of fluid through the contact surface, the flow control rotor 66 may be provided with a protrusion, such as an o-ring or a gasket. The protrusion can be made of a flexible material such as rubber, poly er, silicone and the like, or an inflexible material such as metal, aluminum, plastic, or polymer to prevent the fluid leakage.

The rotating type valve is not limited to the structure shown in the drawings and may be modified into different structures. In addition, the flow control unit 60 is not limited to the structures described above and may be modified into other structures that control a flow through the inflow and outflow tubes.

The blood dialyzing apparatus 1 may also include various safety and monitoring sensors 24 and 34 for blood and dialysis fluid, respectively. The sensors monitor the blood dialyzing treatment and include pressure sensors, air bubble sensor, blood leak sensor, temperature sensor, a conductivity sensor, and the like. An additional filter such as an endotoxin filter may be installed in the circuit of the blood dialyzing apparatus 1 to ensure no harmful substances to come in contact with blood.

Hereinafter, an operation of the blood dialyzing apparatus 1 according to an embodiment of the present invention will be described in detail with reference to the accompanying drawings.

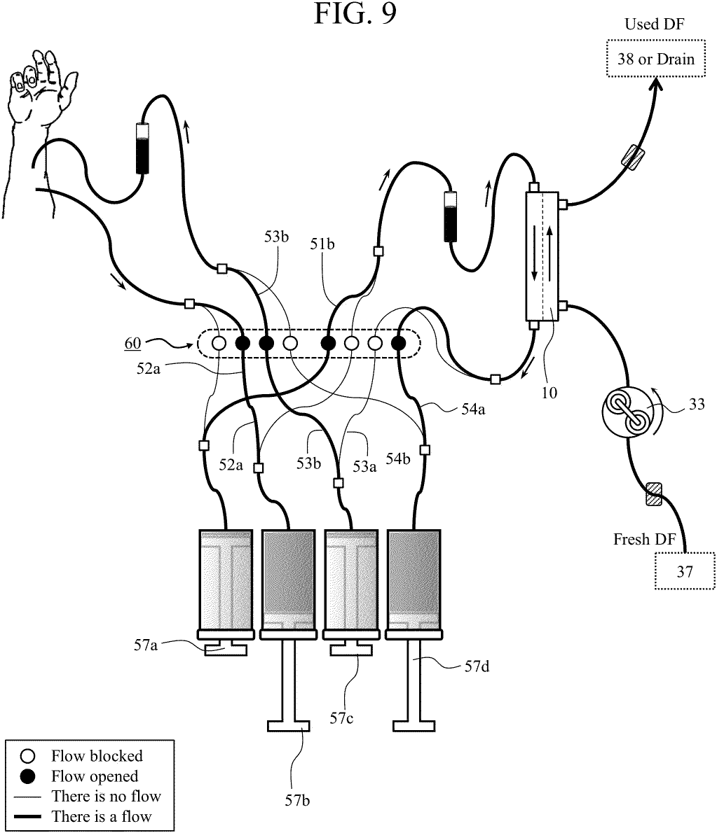

As shown in FIG. 9, the first and third chambers 51 and 53 are compressed and the second and fourth chambers 52 and 54 are expanded. The flow control unit 60 blocks flow passages through the tubes 51a, 52b, 53a and 54b, and opens through the tubes 51b, 52a, 53b and 54a.

Due to the compression of the first chamber 51, blood in the chamber is provided to the blood dialyzing filter 10 through the first chamber outflow tube 51b.

Due to the expansion of the second chamber 52, blood of the patient flows into the chamber 52 through the second chamber inflow tube 52a.

Due to the compression of the third chamber 53, blood in the chamber is returned to the patient through third chamber outflow tube 53b.

Due to the expansion of the fourth chamber 54, blood of the blood dialyzing filter 10 flows into the chamber 54 through the fourth chamber inflow tube 54a.

This is termed a Phase A.

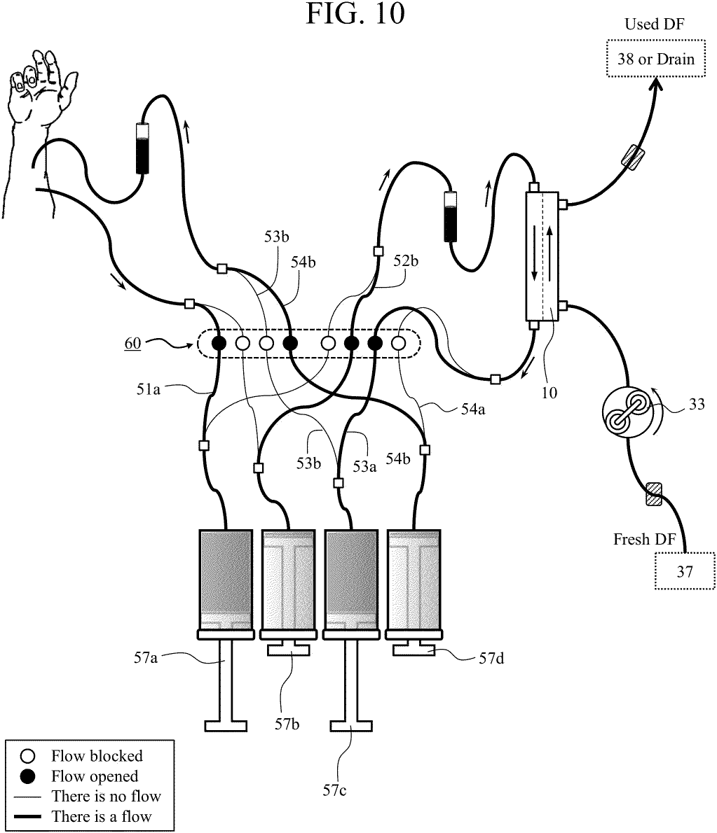

On the other hand, as shown in FIG. 10, the first and third chambers 51 and 53 are expanded and the second and fourth chambers 52 and 54 are compressed. The flow control unit 60 opens flow passages through the tubes 51a, 52b, 53a and 54b, and blocks flow passages through the tubes 51b, 52a, 53b and 54a.

Due to the expansion of the first chamber 51, blood of the patient is supplied to the chamber through the first chamber inflow tube 51a.

Due to the compression of the second chamber 52, blood inside the chamber is supplied to the blood dialyzing filter 10 through the second chamber outflow tube 52b.

Due to the expansion of the third chamber 53, blood of the blood dialyzing filter 10 fills the chamber 53 through the third chamber inflow tube 53a.

Due to the compression of the fourth chamber 54, blood of the chamber is returned to the patient through the fourth chamber outflow tube 54b.

This state is termed a Phase B.

The blood dialyzing apparatus 1 according to an embodiment of the present invention repeats Phases A and B. During the Phase A, the first chamber 51 supplies blood to the blood dialyzing filter 10 but the second chamber 52 does during Phase B. The first and second chambers alternately supply blood to the blood dialyzing filter 10, so they servs as supplying chambers. The expression ‘first’ and ‘second’ chambers are merely used to describe the ‘two’ blood supplying chambers. When the first chamber is compressed and the second chamber is expanded, or vice versa, it means that one of the supplying chambers 51 and 52 is compressed while the other one is expanded.

The third and fourth chambers 53 and 54 return blood of the blood dialyzing filter 10 to a patient, so they are discharging chambers. When the third chamber is compressed and the fourth chamber is expanded, or vice versa, it means one of the discharging chambers is compressed and the other one expands.

The blood dialyzing apparatus 1 according to an embodiment of the present invention may be modified into a structure in which the chambers 51 to 54 transfer dialysis fluid through the blood dialyzing filter 10. The first and second chambers alternately supply dialysis fluid to the blood dialyzing filter 10, so they servs as dialysis fluid supplying chambers.

Likewise, the third and fourth chambers 53 and 54 discharge dialysis fluid from the blood dialyzing filter 10, so they serve as the dialysis fluid discharging chambers. Substantially the same description as shown in FIGS. 9 and 10 is applied to the operation of the blood dialyzing apparatus 1 when the chambers 51 to 54 transfer dialysis fluid through the blood dialyzing filter 10.

Therefore, the first and second chambers serve as the supplying chambers of the blood or dialysis fluid while the third and fourth chambers are the discharging chambers of the same.

Hereinafter, a method of operating the blood dialyzing apparatus 1 according to an embodiment of the present invention will be described in detail with reference to the accompanying drawings.

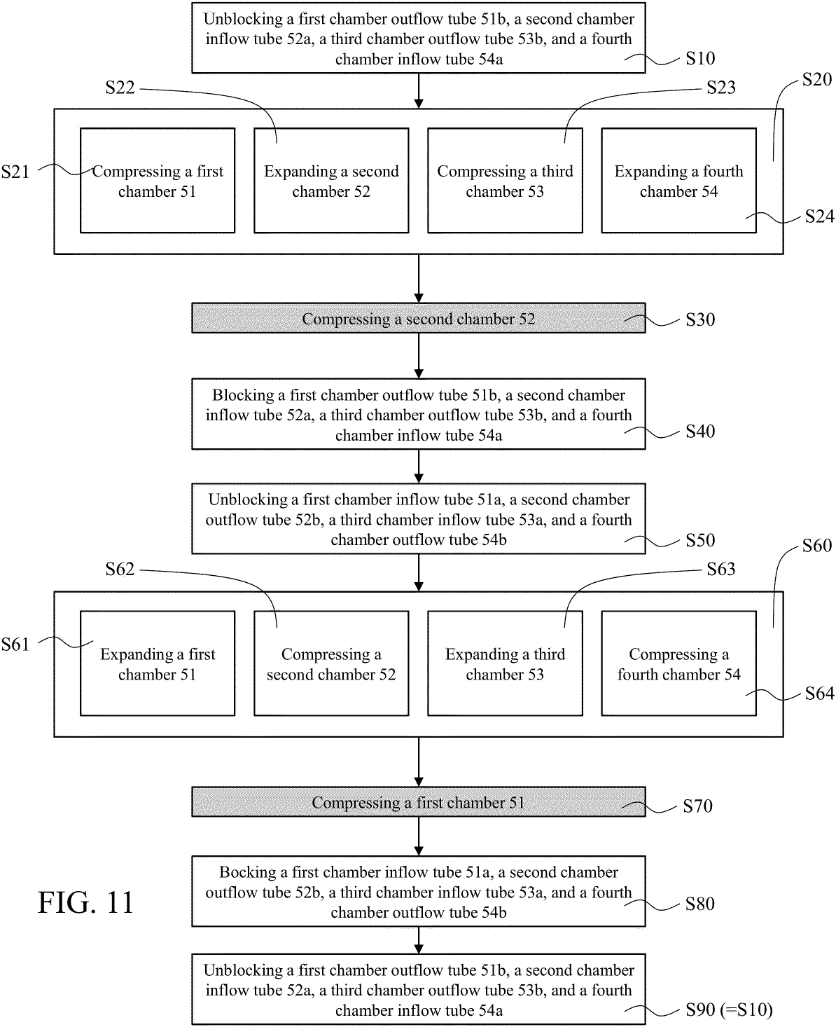

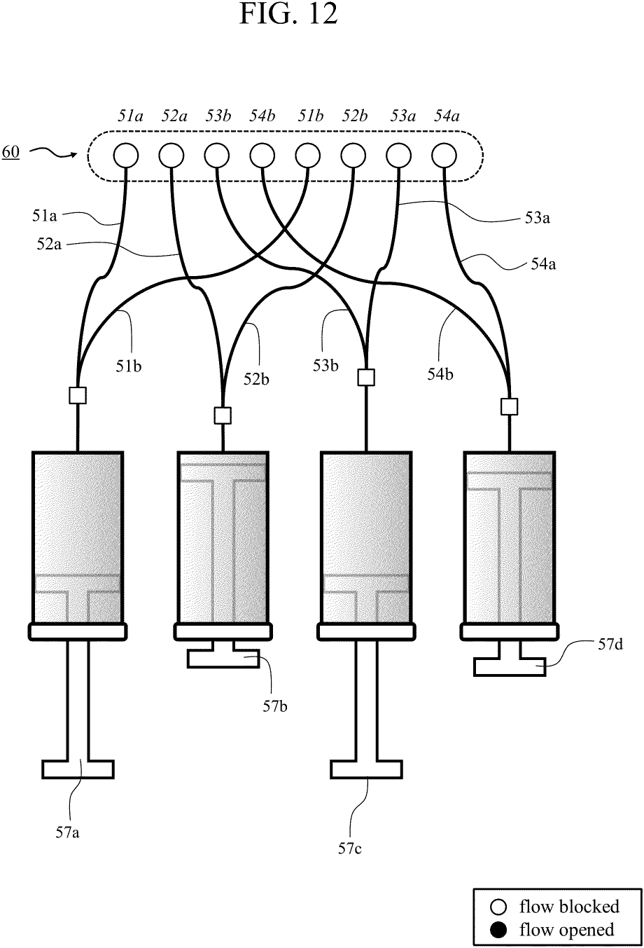

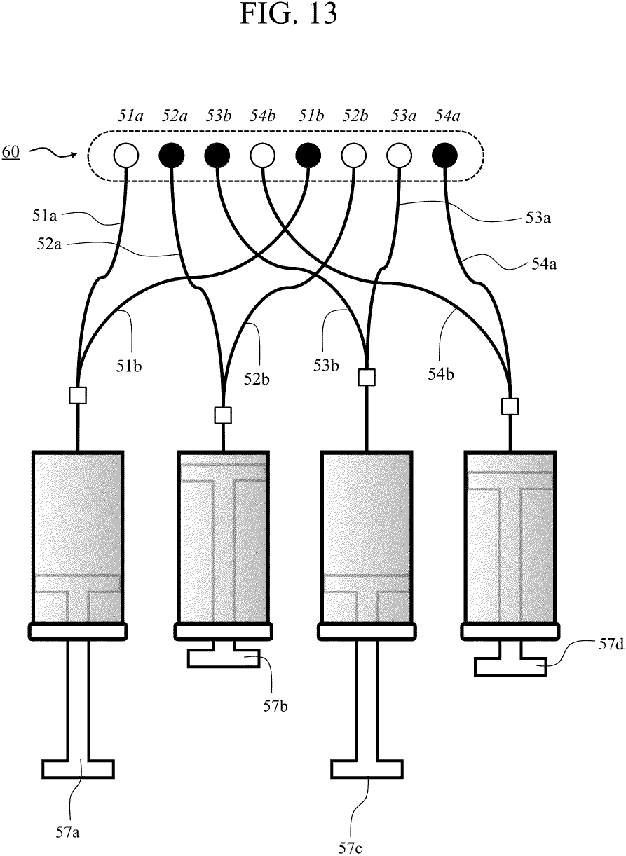

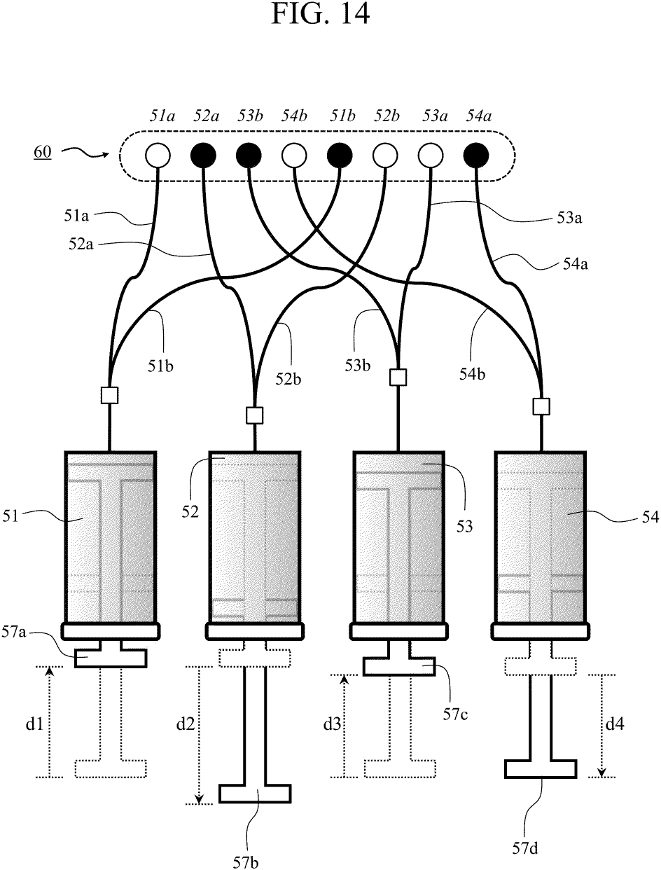

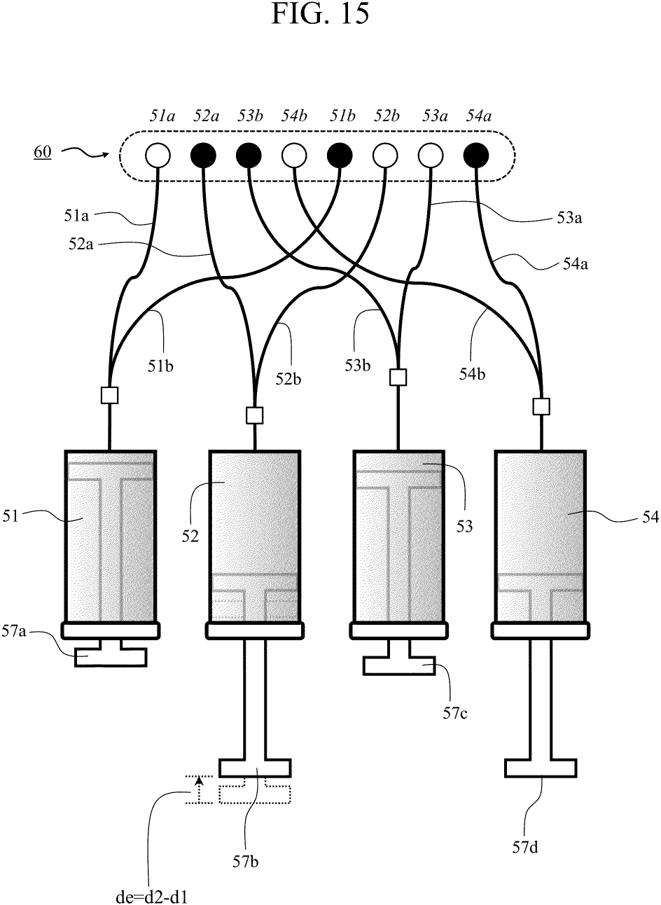

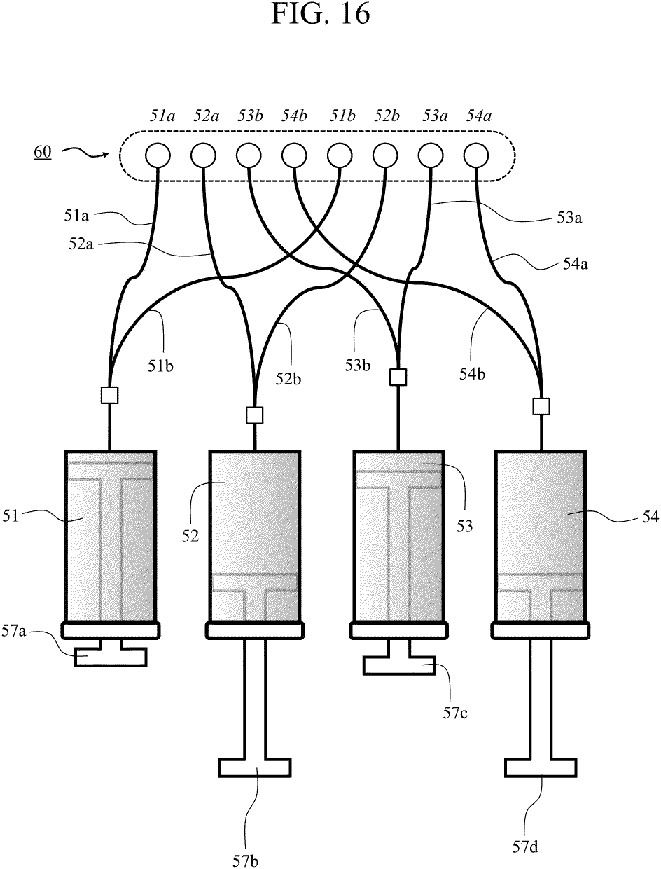

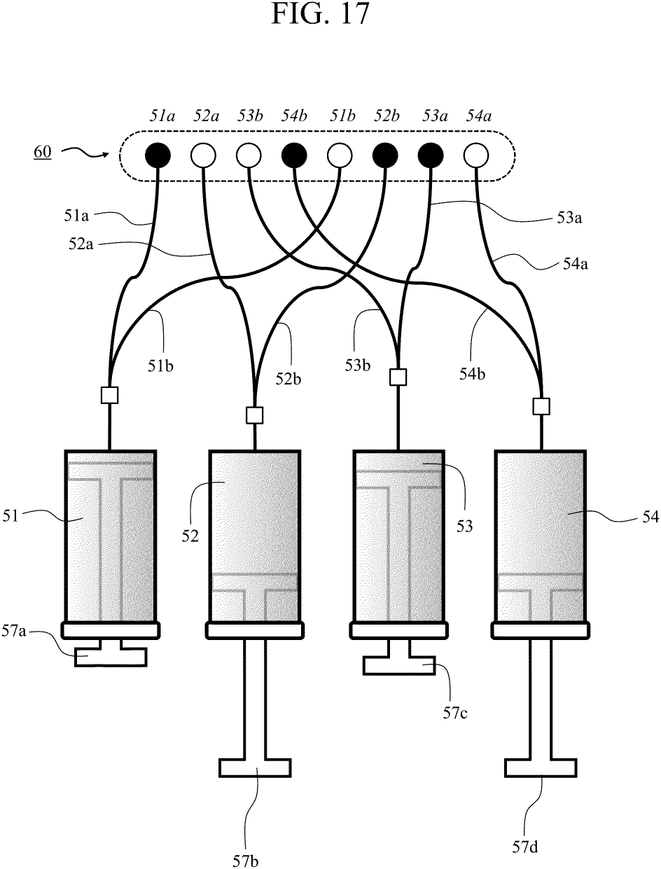

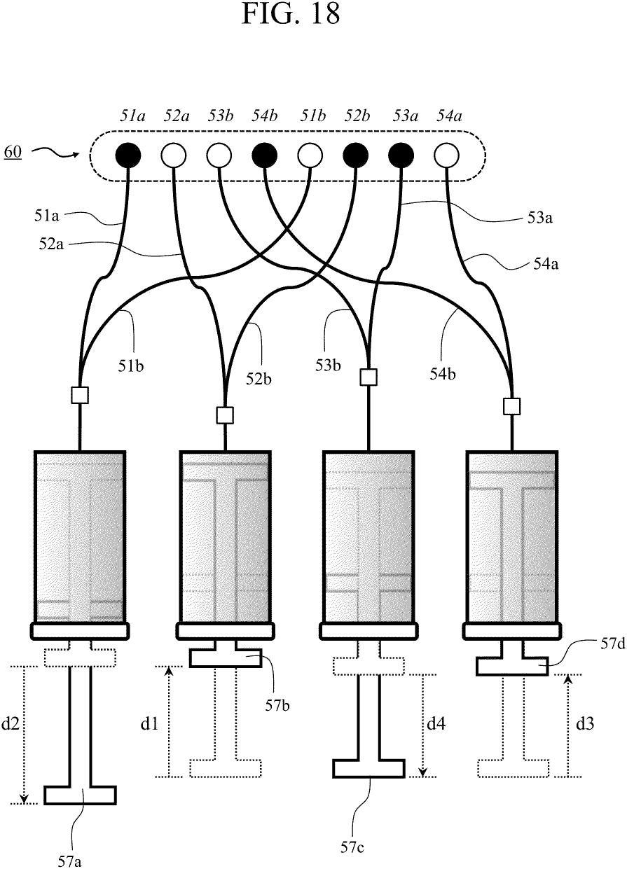

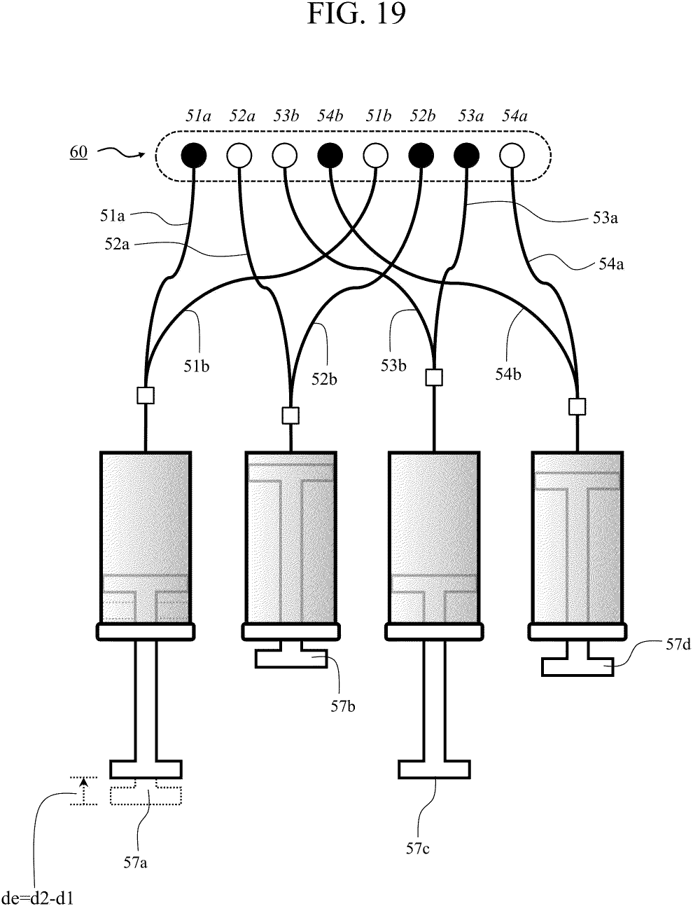

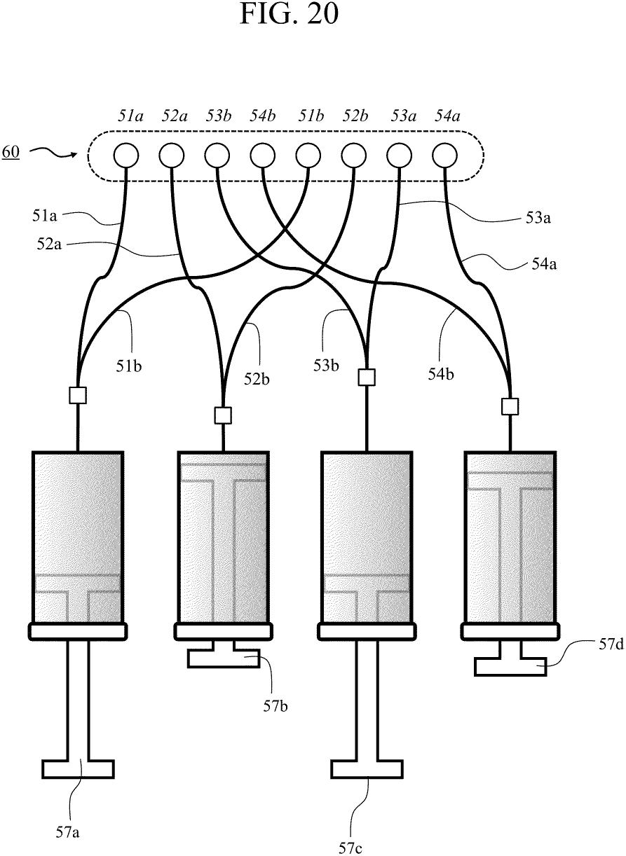

FIG. 11 is a flowchart showing the steps of operating the blood dialyzing apparatus 1. The steps include S10 to S90, which are illustrated in the drawings as summarized in the table below.

| | | |
|---|---|---|
| Step | Reference Drawings | Operation |
| S10 | FIG. 12 to FIG. 13 | Operating the flow control unit 60 |
| S20 (S21∼S24) | FIG. 13 to FIG. 14 | |
| S30 | FIG. 14 to FIG. 15 | |
| S40 | FIG. 15 to FIG. 16 | Operating the flow control unit 60 |
| S50 | FIG. 16 to FIG. 17 | Operating the flow control unit 60 |
| S60 (S61∼S64) | FIG. 17 to FIG. 18 | |
| S70 | FIG. 18 to FIG. 19 | |
| S80 | FIG. 19 to FIG. 20 | Operating the flow control unit 60 |
| S90 | FIG. 12 to FIG. 13 | Operating the flow control unit 60 |

In an embodiment, the blood dialyzing apparatus 1 may be configured to repeat the steps S10 to S80 which constitute one cycle.

### S20 (S21 to S24, With Reference to FIG. 14)

The steps S21 to S24 involve the movement of the chamber pressurizing members 57a to 57d to compress the first chamber 51 (S21), expand the second chamber 52 (S22), compress the third chamber 53 (S23), and expand the fourth chamber 54 (S24). For example, the S21 to S24 may represent the Phase A shown in FIG. 9, where the second chamber 52 expands to draw blood from a patient while the first chamber 51 is compressed and supplies blood to the blood dialyzing filter 10. Also, the third chamber 53 is compressed to supply blood to a patient and the fourth chamber 54 expands to draw blood from the blood dialyzing filter 10.

Since the chambers are expanded or compressed, the expanded volume or the compressed volume of the chamber may be determined, which is termed a stroke volume (SV) of the chamber.

For example, when the first and second chamber pressurizing members 57a and 57b are driven by a single chamber pressurizing member (or the first and second chamber pressurizing members 57a and 57b are formed in a single body), the stroke volumes of the first and second chambers 51 and 52 may be equally maintained. The same description may be applied to the third and fourth chamber pressurizing members 57c and 57d.

However, according to an embodiment of the present invention, when the first chamber 51 is compressed and the second chamber 52 is expanded, the SV of the supplying chamber that is expanded (i.e., SV52) is equal to or greater than the SV of the supplying chamber that is compressed (i.e., SV51). Here, SV51 to SV54 are the stroke volumes of the chambers 51 to 54, respectively, either compressed or expanded.

Specifically, the expanded SV of the supplying chamber may be greater than the compressed SV of the supplying chamber by 2% to 60% of the compressed SV of the supplying chamber. Preferably, the expanded SV of the supplying chamber is greater than the compressed SV of the supplying chamber by 6% to 18% of the compressed SV of the supplying chamber. More desirably, by 8% to 12% of the compressed SV of the supplying chamber.

In a similar manner, when the third chamber 53 is compressed and the fourth chamber 54 is expanded, the SV of the discharging chamber that is expanded (i.e., SV54) may be equal to or greater than the SV of the discharging chamber that is compressed (SV53).

Specifically, the expanded SV of the discharging chamber may be greater than the compressed SV of the discharging chamber by 2% to 60% of the compressed SV of the discharging chamber. Preferably, the expanded SV of the discharging chamber is greater than the compressed SV of the discharging chamber by 6% to 18% of the compressed SV of the discharging chamber. More desirably, by 8% to 12% of the compressed SV of the discharging chamber.

In addition, the compressed volume of the supplying chamber is configured to have a different value from the expanded volume of the discharging chamber. For example, with reference to FIGS. 9 and 10, the compressed volume of the first chamber 51 may be larger than the expanded volume of the fourth chamber 54 during Phase A. The compressed volume of the second chamber 52 may be equal to or greater than the expanded volume of the third chamber 53 during Phase B.

Referring to FIG. 14, when the chamber is made of a substantially inflexible material having a cylinder shape with a unform inner diameter, the stroke volumes of the chamber - either compressed or expanded - vary according to the distance that the chamber pressurizing members 57a to 57d move. For example, the stoke volume can be determined by the chamber radius (R) and the length (d) the chamber pressurizing member travels, as follows.
SV51 = π*R1²*d1
SV52 = π*R2²*d2
SV53 = π*R3²*d3
SV54 = π*R4²*d4

Where, R1 to R4 are the radius of the chambers 51 to 54, respectively.

Here, the d1 is the distance of the chamber pressurizing member which compresses one supplying chamber. The d2 is the distance of the chamber pressurizing member which expands another supplying chamber. The d3 is the distance of the chamber pressurizing member which compresses one discharging chamber, and d4 is the distance of the chamber pressurizing member which expands another discharging chamber. When R1 to R4 are equal, SV51 to SV54 are determined by d1 to d4.

One of the supplying chambers is compressed and fluid in the chamber is supplied to the blood dialyzing filter 10. Simultaneously, one of the discharging chambers expands and the fluid of the blood dialyzing filter 10 is discharged to the chamber. The difference in the stroke volumes between the supplying chamber and the discharging chamber generates water flux across the membranes 12. The blood dialyzing apparatus 1 according to an embodiment of the present invention is capable of regulating the amount of water flux across membranes 12 – either blood to dialysis fluid or dialysis fluid to blood.

### S30 (with Reference to FIG. 15)

The blood dialyzing apparatus 1 according to an embodiment of the present invention may further involve a reverse movement of one or more chamber pressurizing members 57a, 57b, 57c or 57c. For example, in FIG. 15, the second chamber pressurizing member 57b moves upward by a predetermined distance of de, whereby the second chamber 52 is slightly compressed. Here, de is preferably smaller than d1 or d2. The de may be set to a difference between d1 and d2.

The short reverse compression of the supplying chamber, promptly following its expansion, is particularly helpful in that the supplying chamber restores the hydraulic pressure inside to a desired range. The reverse movement of the second chamber pressurizing member 57b (S30) occurs before the blocking of the flow control unit 60 (S40).

FIG. 15 illustrates the reverse compression of the supplying chamber after its expansion. However, the reverse movement is not limited to the supply chamber and may be applied to the discharging chamber. For example, the fourth chamber pressurizing member 57d may move upward to compress the fourth chamber 54 after it was expanded previously.

### S60 (S61 to S64, With Reference to FIG. 18)

Substantially the same description used for the steps S21 to S24 is applied for the steps S61 to S64 except that the compression and expansion of the chambers 51 to 54 are reversed. For example, the steps S61 to S64 illustrated in FIG. 18 represent Phase B of FIG. 10, in which the first chamber 51 expands and the second chamber 52 is compressed.

### S70 (with Reference to FIG. 19)

Substantially the same description used for the step S30 is applied to the step S70.

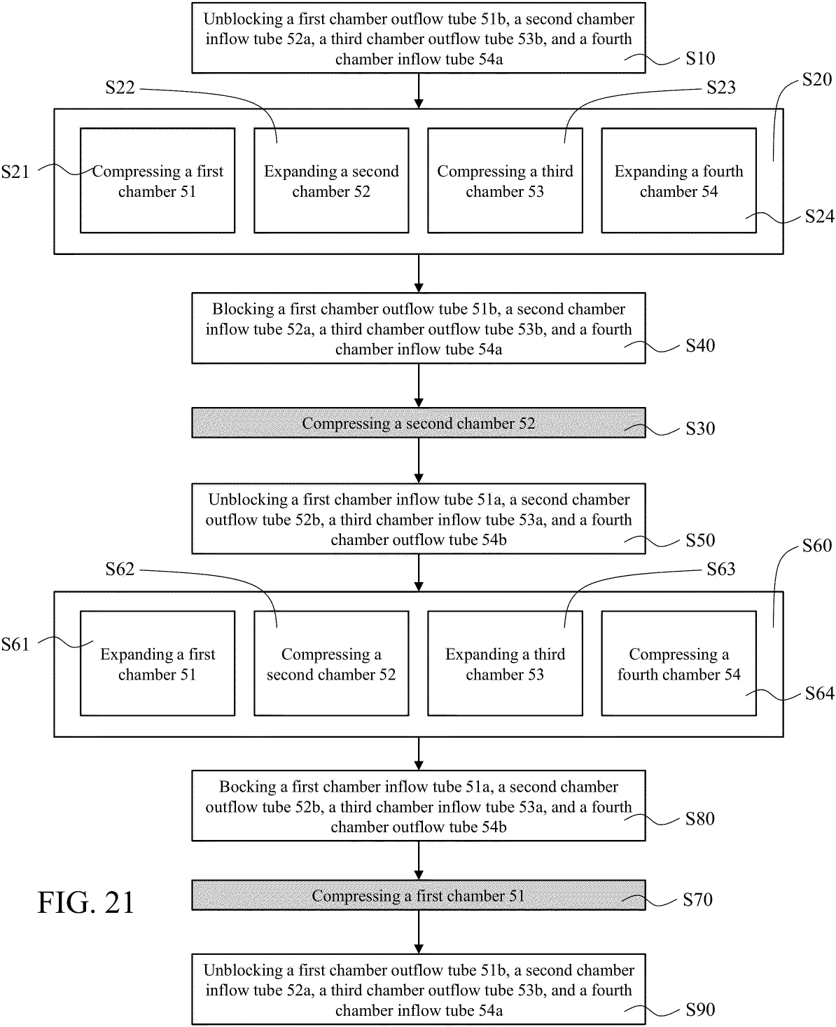

In addition, the operation of the blood dialyzing apparatus 1 is not limited to the steps shown in FIG. 11. The blood dialyzing apparatus 1 may repeat the steps S10 to S80, but the sequence may be modified. Exemplary sequence of the operating steps is illustrated in FIG. 21, where S30 is conducted after S40 and S80 precedes S70. The sequence of each step according to an embodiment of the present invention may further be modified to ensure the stable and efficient operation of the dialyzing apparatus.

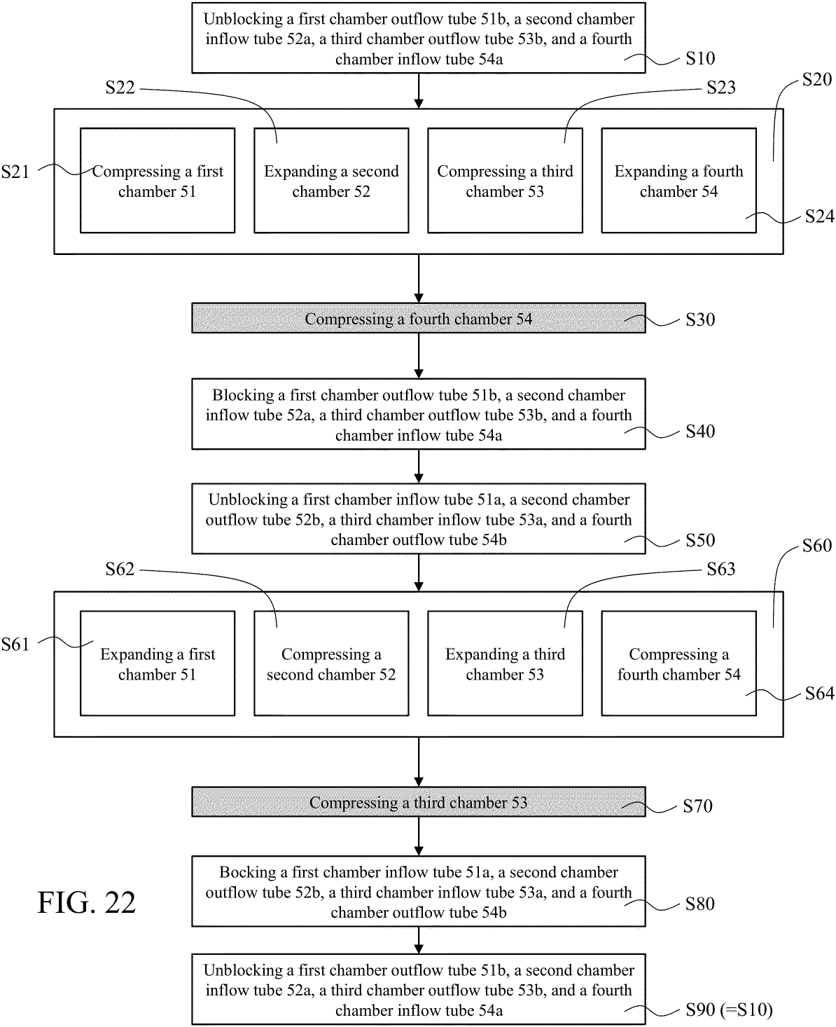

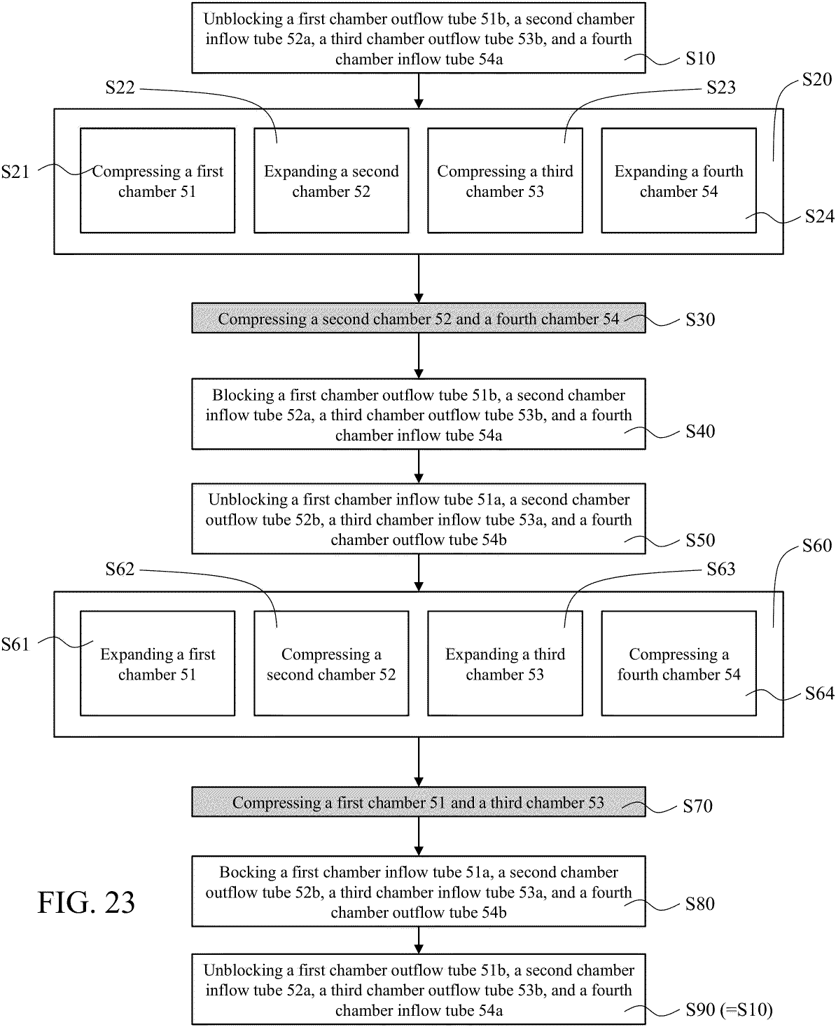

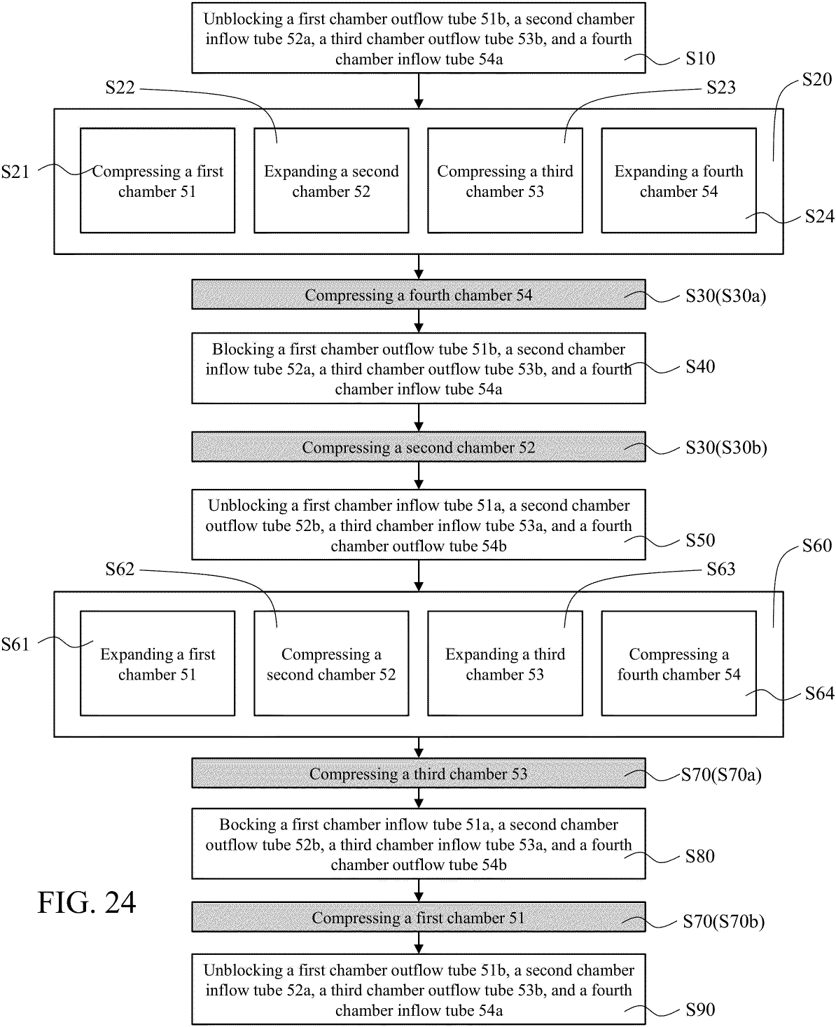

The step S30 is not limited to the drawings, and it may be modified as shown in FIGS. 22 and 23. For example, S30 may involve the short compression of the discharging chamber that was previously expanded, not merely for the supplying chambers. Also, the operation of the blood dialyzing apparatus 1 may employ the short reverse movements of the multiple chamber pressurizing members at S30. Another embodiment for the operation is also illustrated in FIG. 24, where S30 may be conducted before and after S40.

In addition, the operation of the blood dialyzing apparatus 1 may further be embodied to include a predetermined amount of time delay between each step. As an example, referring to FIG. 11, it may be necessary to pause for a predetermined time after S20 (i.e., each of S21 to S24) and S60 (i.e., each of S61 to S64), that is, inserting a time delay to ensure the hydraulic pressure of the chamber recovered to a preset range. In an embodiment, the time delay after S20 or S60 may be set to a value between 0.2 to 2.8 seconds, more preferably 0.8 to 1.6 seconds. In a similar manner, a time delay may further be included after S30 and S40 (or S70 and S80), which ranges between 0.1 to 1.2 seconds.

S21 to S24 may take substantially the same amount of time, ranging 1.5 to 6.5 seconds, which is similarly applied to S61 to S64. Likewise, the steps S30 and S70 take substantially the same amount of time.

However, the operation of the blood dialyzing apparatus 1 is not limited thereto and may modified. The time period for S21 to S24 (or the time period for S61 to S64) may be set at different values. In an embodiment, the compression of the supplying chamber (e.g., S21) may take shorter than the expansion of the discharging chamber (e.g., S24). The compression of the supplying chamber may take a time that is equal to 40% to 80% of the expansion of the discharging chamber. In other words, when the expansion of the discharging chamber takes 6 seconds, the compression of the supplying chamber is configured to be done in 2.4 seconds to 4.8 seconds.

Alternatively, the expansion of the discharging chamber may take shorter than the compression of the supplying chamber. For example, when the compression of the supplying chamber takes 6 seconds, the expansion of the discharging chamber may take 2 to 6 seconds.

Substantially the same description can also be applied to the first and second chambers (i.e., between the supplying chambers) and the third and fourth chambers (i.e., between the discharging chambers). S21 takes shorter than S22, or vice versa. S23 takes shorter than S24, or vice versa.

Preferably, S21, S22, S23 or S24 may take longer than S30, S40, or S50. While S21 to S24 take 1.5 to 6.5 seconds, S30, S40 or S50 may take 0.1 to 2.4 seconds.

The blood dialyzing apparatus 1 uses the chambers 51 to 54 as the means of transferring blood and they are connected to the blood dialyzing filter 10 and the patient (FIGS. 2A and 2B). However, the blood dialyzing apparatus 1 according to an embodiment of the present invention is not limited thereto, and obviously can be modified into a structure, for example where the chambers 51 to 54 transfer dialysis fluid through the blood dialyzing filter 10 (FIG. 2C). In addition, the dialysis fluid pump 33 shown in FIGS. 2A and 2B may be modified to a blood pump 23.

The dialysis fluid pump 33 or the blood pump 23 is illustrated with a peristaltic roller pump in the drawing, but the pumps 23 and 33 are not limited to the peristaltic pump. Any types of volume displacement pumps may be used for the pump 33, including but not limited to a gear pump, a lobe pump, a rotary piston pump, a piston pump, or the like.

Provided is the blood dialyzing apparatus according to an embodiment of the present invention, in which multiple blood chambers are compressed and expanded to transfer blood. The multiple chambers ensure blood flow rates upstream and downstream of the blood dialyzing filter to be regulated independently. Neither separate blood pump nor a separate ultrafiltration pump, nor a separate balancing chamber are required. Therefore, the entire system can be sufficiently miniaturized and light-weighted, and easy to be installed while reducing the cost for blood dialyzing treatment. The blood dialyzing apparatus will eventually be an optimal alternative for the blood dialyzing treatment in a place out of hospitals.

The above-disclosed subject matter is to be considered illustrative, and not restrictive, and the appended claims are intended to cover all such modifications, enhancements, and other embodiments, which fall within the true spirit and scope of the present invention. Thus, to the maximum extent allowed by law, the scope of the present invention is to be determined by the broadest permissible interpretation of the following claims and their equivalents, and shall not be restricted or limited by the foregoing detailed description.

What is claimed is:

1. A blood dialyzing apparatus comprising:
a blood dialyzing filter;
first to fourth chambers each having an internal space;
first to fourth chamber pressurizing members compressing or expanding the internal space of the respective first to fourth chambers;
a chamber pressurizing member driver operating the chamber pressurizing members; and
a flow control unit, wherein
the first to fourth chambers are each connected with an inflow tube and an outflow tube, wherein a fluid is provided to the chamber through the inflow tube and the fluid of the chamber is discharged therefrom through the outflow tube,
the outflow tubes of the first and second chambers are connected to the blood dialyzing filter and the inflow tubes of the third and fourth chambers are connected to the blood dialyzing filter,
the fluid is supplied to the blood dialyzing filter through the first and second chambers and the fluid of the blood dialyzing filter is discharged therefrom through the third and fourth chambers, and
the flow control unit opens or blocks a flow through the inflow and outflow tubes connected to the first to fourth chambers.

2. The blood dialyzing apparatus of claim 1, wherein the first chamber is compressed when the second chamber is expanded, and the third chamber is compressed when the fourth chamber is expanded.

3. The blood dialyzing apparatus of claim 2, wherein an expanded volume of the second chamber is equal to or greater than a compressed volume of the first chamber.

4. The blood dialyzing apparatus of claim 3, wherein the expanded volume of the second chamber is greater than the compressed volume of the first chamber by 2% to 60% of the compressed volume of the first chamber.

5. The blood dialyzing apparatus of claim 3, wherein an expanded volume of the fourth chamber is equal to or greater than a compressed volume of the third chamber.

6. The blood dialyzing apparatus of claim 5, wherein the expanded volume of the fourth chamber is greater than the compressed volume of the third chamber by 2% to 60% of the compressed volume of the third chamber.

7. The blood dialyzing apparatus of claim 5, wherein the compressed volume of the first chamber is greater than the expanded volume of the fourth chamber by 2% to 26% of the expanded volume of the fourth chamber.

8. The blood dialyzing apparatus of claim 5, wherein the expanded volume of the fourth chamber is greater than the compressed volume of the first chamber by 2% to 26% of the compressed volume of the first chamber.

9. A method of operating a blood dialyzing apparatus comprising first to fourth chambers each having an internal space and connected with an inflow tube and an outflow tube, wherein a fluid is provided to the chamber through the inflow tube and the fluid of the chamber is discharged therefrom through the outflow tube, wherein the first and second chambers supply the fluid to a blood dialyzing filter while the third and fourth chambers discharge the fluid from the blood dialyzing filter, the method comprising steps of:
unblocking the outflow tube of the first chamber, the inflow tube of the second chamber, the outflow tube of the third chamber and the inflow tube of the fourth chamber (S10);
compressing the first chamber to discharge the fluid therein to the blood dialyzing filter (S21);
expanding the second chamber to allow the fluid to flow therein (S22);
compressing the third chamber to discharge the fluid therefrom (S23); and
expanding the fourth chamber to allow the fluid of the blood dialyzing filter to flow into the chamber (S24).

10. The method of claim 9, wherein the compressing of the first chamber S21, the expanding of the second chamber S22, the compressing of the third chamber S23, and the expanding of the fourth chamber S24 occur substantially at the same time,
wherein the compressing of the first chamber S21, the expanding of the second chamber S22, the compressing of the third chamber S23, and the expanding of the fourth chamber S24 take 1.5 to 6.5 seconds.

11. The method of claim 10, wherein an expanded volume of the second chamber is equal to or greater than a compressed volume of the first chamber.

12. The method of claim 10, wherein the expanded volume of the second chamber is greater than the compressed volume of the first chamber by 2% to 60% of the compressed volume of the first chamber.

13. The method of claim 11, wherein an expanded volume of the fourth chamber is equal to or greater than a compressed volume of the third chamber.

14. The method of claim 13, wherein the expanded volume of the fourth chamber is greater than the compressed volume of the third chamber by 2% to 60% of the compressed volume of the third chamber.

15. The method of claim 13, wherein the compressed volume of the first chamber is greater than the expanded volume of the fourth chamber by 2% to 26% of the expanded volume of the fourth chamber.

16. The method of claim 13, wherein the expanded volume of the fourth chamber is greater than the compressed volume of the first chamber by 2% to 26% of the compressed volume of the first chamber.

17. The method of claim 13, further comprising a step of compressing the second chamber (S30), wherein S30 follows S21, and a compressed volume of the second chamber at S30 is smaller than the expanded volume of the second chamber at S22 or the compressed volume of the first chamber at S21.

18. The method of claim 13, further comprising a step of compressing the fourth chamber (S30), wherein S30 follows S24, and a compressed volume of the fourth chamber at S30 is smaller than the expanded volume of the fourth chamber at S24 or the compressed volume of the third chamber at S23.

19. The method of claim 13, further comprising steps of:
blocking the outflow tube of the first chamber, the inflow tube of the second chamber, the outflow tube of the third chamber, and the inflow tube of the fourth chamber (S40);
unblocking the inflow tube of the first chamber, the outflow tube of the second chamber, the inflow tube of the third chamber and the outflow tube of the fourth chamber (S50);
expanding the first chamber to allow the fluid to flow into the chamber (S61);
compressing the second chamber to supply the fluid to the blood dialyzing filter (S62);
expanding the third chamber to allow the fluid of the blood dialyzing filter to flow into the chamber (S63); and
compressing the fourth chamber to discharge the fluid therefrom (S64).

20. The method of claim 13, further comprising steps of:
blocking the outflow tube of the first chamber, the inflow tube of the second chamber, the outflow tube of the third chamber, and the inflow tube of the fourth chamber (S40);
compressing the second chamber (S30);
unblocking the inflow tube of the first chamber, the outflow tube of the second chamber, the inflow tube of the third chamber and the outflow tube of the fourth chamber (S50);
expanding the first chamber to allow the fluid to flow into the chamber (S61);
compressing the second chamber to supply the fluid to the blood dialyzing filter (S62);
expanding the third chamber to allow the fluid of the blood dialyzing filter to flow into the chamber (S63); and
compressing the fourth chamber to discharge the fluid therefrom (S64).
